(19) United States
(12) Patent Application Publication
(10) Pub. No.: US 2007/0054868 A1
(43) Pub. Date: Mar. 8, 2007

(54) Synergistic polyphenol compounds, compositions thereof, and uses thereof

(75) Inventor: I. Bernard Weinstein, New York, NY (US)

Correspondence Address: WilmerHale/Columbia University, 399 PARK AVENUE, NEW YORK, NY, 10022, US

(73) Assignee: The Trustees of Columbia University in the City of New York, New York, NY (US)

(21) Appl. No.: 11/471,343

(22) Filed: Jun. 20, 2006

Related U.S. Application Data
(60) Provisional application No. 60/692,108, filed on Jun. 20, 2005.
(60) Provisional application No. 60/787,305, filed on Mar. 29, 2006.

(51) Int. Cl. A61K 31/7072; A61K 31/7048; A61K 31/353; A61K 31/337

(52) U.S. Cl. 514/27; 514/49; 514/449; 514/456

(57) ABSTRACT

The present invention relates to Synergistic Polyphenol Compounds, compositions thereof, and methods for treating or preventing cancer in a subject, the methods comprising administering to a subject an effective amount of a Synergistic Polyphenol Compound or composition thereof.

This application claims the benefit under 35 U.S.C. §119(e) of U.S. Provisional Patent Application No. 60/692,108, filed Jun. 20, 2005, and U.S. Provisional Patent Application No. 60/787,305, filed Mar. 29, 2006, each of which are hereby incorporated by reference herein in their entirety.

All patents, patent applications and publications cited herein are hereby incorporated by reference in their entirety. The disclosures of these publications in their entireties are hereby incorporated by reference into this application in order to more fully describe the state of the art as known to one skilled therein as of the date of the invention described and claimed herein.

This patent disclosure contains material that is subject to copyright protection. The copyright owner has no objection to the facsimile reproduction by anyone of the patent document or the patent disclosure as it appears in the U.S. Patent and Trademark Office patent file or records, but otherwise reserves any and all copyright rights.

### 1. FIELD OF THE INVENTION

The present invention relates to Synergistic Polyphenol Compounds, compositions thereof, and methods for treating or preventing cancer in a subject, the methods comprising administering to a subject an effective amount of a Synergistic Polyphenol Compound or composition thereof.

### 2. BACKGROUND OF THE INVENTION

Cancer is second only to cardiovascular disease as a cause of death in the United States. The American Cancer Society estimated that in 2002, there were 1.3 million new cases of cancer and 555,000 cancer-related deaths. There are currently over 10 million living Americans who have been diagnosed with cancer and the NIH estimates the direct medical costs of cancer as over $100 billion per year with an additional $100 billion in indirect costs due to lost productivity—the largest such costs of any major disease.

Modalities useful in the treatment of cancer include chemotherapy, radiation therapy, surgery and biological therapy (a broad category that includes gene-, protein- or cell-based treatments and immunotherapy).

Despite the availability to the clinician of a variety of anticancer agents, traditional chemotherapy has many drawbacks. Almost all anticancer agents are toxic, and chemotherapy can cause significant, and often dangerous, side effects, including severe nausea, bone marrow depression, liver, heart and kidney damage, and immunosuppression. Additionally, many tumor cells eventually develop multi-drug resistance after being exposed to one or more anticancer agents. As such, single-agent chemotherapy is curative in only a very limited number of cancers. Most chemotherapeutic drugs act as anti-proliferative agents, acting at different stages of the cell cycle. Since it is difficult to predict the pattern of sensitivity of a neoplastic cell population, or the current stage of the cell cycle that a cell happens to be in, it is common to use multi-drug regimens in the treatment of cancer, which are typically more effective, but also more toxic than single-drug chemotherapy regimens.

The EGFR subfamily of RTKs includes, in addition to EGFR and HER2, the receptors HER3 (erbB3) and HER4 (erbB4). Binding of specific ligands to EGFR, HER3, and HER4 results in receptor homo- and heterodimerization thus activating the tyrosine kinase activities of these receptors, including HER2 which lacks its own ligand binding domain. This leads to activation of downstream signaling pathways including the MAPK and PI3K pathways, and the expression of genes that enhance cell proliferation. Aberrant expression and constitutive activation of members of the EGFR family have been observed in several types of human malignancies, including colorectal cancer. Thus, human colorectal carcinoma often displays overexpression of both EGFR and HER2. Increased levels of expression of HER3 mRNA and protein are also frequently seen in colorectal carcinoma. Although HER3 does not itself possess an active kinase, the HER2/HER3 heterodimer can play a critical role in enhancing the growth of cancer cells.

It is known that the HER2/HER3 pathway in colon cancer cells induces the expression of COX-2 mRNA and protein, and results in the accumulation of PGE₂, a major metabolic product of COX-2. COX-2 expression is also induced by several oncogenes, tumor promoters, cytokines, and growth factors. In addition, COX-2 expression increases during colon carcinogenesis and it is frequently overexpressed in human colon carcinoma. Although the precise mechanism by which increased expression of COX-2 enhances colon carcinogenesis is not known, it is possible that the production of PGE₂ by this enzyme plays an important role, since it is known that treatment of colon cancer cells with PGE₂ results in transactivation of the EGFR, activation of ERK and stimulation of cell proliferation.

Previous studies indicate that the naturally occurring polyphenol EGCG can inhibit activation of the tyrosine kinase activities of several RTKs including EGFR, HER2, PDGFR, and FGFR. In addition to acting on cell surface receptors, EGCG can also directly target intracellular signaling molecules. Thus, EGCG can directly inhibit the subcellular kinase activities of ERK and Akt in extracts of immortalized cervical cells.

There is increasing evidence for a complex positive feedback circuitry between the EGFR signaling system, COX-2, and PGE₂, which can stimulate the growth of colon cancer cells. Thus, activation of the EGFR and related RTKs leads to the induction of COX-2 expression and the synthesis of PGE₂. Treatment of colon cancer cells with PGE₂, in turn, leads to rapid phosphorylation of the EGFR and ERK activation, and stimulation of cell growth. On the other hand, inactivation of EGFR kinase with selective inhibitors causes a decrease in PGE₂-induced ERK activation and cell proliferation. However, the precise mechanisms by which PGE₂ leads to activation of the EGFR and the RAS/MAPK pathway are not currently known.

Amongst the members of the EGF receptor family, HER3 is the most efficient activator of PI3K since it contains six docking sites for the p85 protein, an adaptor subunit of PI3K. It has been reported that in colon cancer cells PGE₂ led to an increase in cell proliferation and motility via activation of the PI3K/Akt pathway. In addition, activation of HER3 by heregulin strongly induces COX-2 expression and PGE₂ synthesis in colon cancer cells. Further, it has also been reported that oral infusion of a green tea mixture inhibits the development and progression of prostate cancer in the TRAMP mouse model of this disease. This was associated with a reduction in the level of IGF-1 and an increase in IGFBP-3 in both the serum and prostate tissue of the treated mice and these results indicate that a non-toxic dose of green tea polyphenols can affect the IGF/IGF-R system in vivo.

Accordingly, there exists a need for inhibitors of the enzymatic activity of COX-2 for the prevention and treatment of colon cancer and other types of cancer, wherein such inhibitors have improved therapeutic indices. This invention addresses that need.

The recitation of any reference in this application is not an admission that the reference is prior art to this application.

### 3. SUMMARY OF THE INVENTION

In one aspect, the invention provides Synergistic Polyphenol Compositions comprising two or more compounds or pharmaceutically acceptable salts thereof, wherein the compounds are selected from: a catechin, including but not limited to (−)-epicatechin, (−)-epicatechin gallate, (−)-gallocatechin gallate, (−)-epigallocatechin and (−)-epigallocatechin gallate; a phenolic acid, including but not limited to gallic acid, caffeic acid and ellagic acid; a bioflavanoid, including but not limited to an anthocyanin, apigenin, and quercetin; and a complex polyphenol, including but not limited to, a tannin and a lignan, and any combination thereof, wherein the amounts of the two or more compounds are together synergistically effective to treat cancer.

In another aspect, the invention provides Synergistic Polyphenol Compositions consisting essentially of two or more compounds or pharmaceutically acceptable salts thereof, wherein the compounds are selected from: a catechin, including but not limited to (−)-epicatechin, (−)-epicatechin gallate, (−)-gallocatechin gallate, (−)-epigallocatechin and (−)-epigallocatechin gallate; a phenolic acid, including but not limited to gallic acid, caffeic acid and ellagic acid; a bioflavanoid, including but not limited to an anthocyanin, apigenin, and quercetin; and a complex polyphenol, including but not limited to, a tannin and a lignan, and any combination thereof, and wherein the amounts of the two or more compounds are together synergistically effective to treat colorectal cancer or liver cancer.

In a further aspect, the invention provides methods for treating or preventing cancer in a subject, the method comprising administering to the subject an effective amount of a Synergistic Polyphenol Composition.

In still another aspect, the invention provides methods for treating or preventing cancer in a subject, the method comprising: (a) administering to the subject a first compound or pharmaceutically acceptable salt thereof, wherein the first compound is selected from: a catechin, including but not limited to (−)-epicatechin, (−)-epicatechin gallate, (−)-gallocatechin gallate, (−)-epigallocatechin and (−)-epigallocatechin gallate; a phenolic acid, including but not limited to gallic acid, caffeic acid and ellagic acid; a bioflavanoid, including but not limited to an anthocyanin, apigenin, and quercetin; and a complex polyphenol, including but not limited to, a tannin and a lignan, and any combination thereof, and (b) administering to the subject a second compound or pharmaceutically acceptable salt thereof, wherein the second compound is selected from: a catechin, including but not limited to (−)-epicatechin, (−)-epicatechin gallate, (−)-gallocatechin gallate, (−)-epigallocatechin and (−)-epigallocatechin gallate; a phenolic acid, including but not limited to gallic acid, caffeic acid and ellagic acid; a bioflavanoid, including but not limited to an anthocyanin, apigenin, and quercetin; and a complex polyphenol, including but not limited to, a tannin and a lignan, and any combination thereof, wherein the first and second compounds are different and wherein the amounts of the first and second compounds administered are together synergistically effective to treat or prevent cancer.

The invention provides methods for treating or preventing cancer in a subject which comprises administering to a subject an effective amount of a Synergistic Polyphenol Composition or one for more Synergistic Polyphenol Compounds.

The present invention may be understood more fully by reference to the following detailed description and illustrative examples, which are intended to exemplify non-limiting embodiments of the invention.

### 4. BRIEF DESCRIPTION OF THE DRAWINGS

FIG. 1 shows the results of cell viability assays which were used to determine the growth inhibitory effects of a combination of (−)-epigallocatechin gallate and (−)-epicatechin on HT29 human colon cancer cells. The x-axis of FIG. 1A represents the (−)-epigallocatechin gallate concentration, while the y-axis represents cell viability as a percentage. The lines on the graph in FIG. 1A from top to bottom, represent the following: (−)-epicatechin alone, (−)-epicatechin+0.1 μg/mL (−)-epigallocatechin gallate, (−)-epicatechin+1.0 μg/mL (−)-epigallocatechin gallate, (−)-epicatechin+10 μg/mL (−)-epigallocatechin gallate, and (−)-epicatechin+20 μg/mL (−)-epigallocatechin gallate. The x-axis of FIG. 1B represents the (−)-epicatechin concentration, while the y-axis represents cell viability as a percentage. The lines on the graph in FIG. 1B from top to bottom, represent the following: (−)-epigallocatechin gallate alone, (−)-epigallocatechin gallate+1.0 μg/mL (−)-epicatechin, (−)-epigallocatechin gallate+10 μg/mL (−)-epicatechin, (−)-epigallocatechin gallate+50 μg/mL (−)-epicatechin, (−)-epigallocatechin gallate+100 μg/mL (−)-epicatechin.

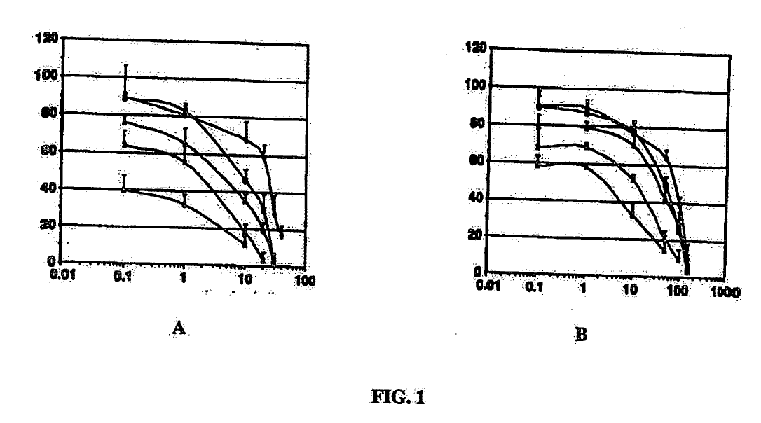

FIG. 2 shows the effects of (−)-epigallocatechin gallate and Poly E on the growth of various human colon cancer cell lines. The x-axis of FIG. 2A represents the relative cell viability as a percentage, while the y-axis represents the concentration of (−)-epigallocatechin gallate in μg/mL. The lines on the graph in FIG. 2A from top to bottom, represent the following human colon cancer cell lines: Caco2, HCT116, HT29, SW480, SW837 and FHC. The x-axis of FIG. 2B represents the relative cell viability as a percentage, while the y-axis represents the concentration of (−)-epigallocatechin gallate in μg/mL.

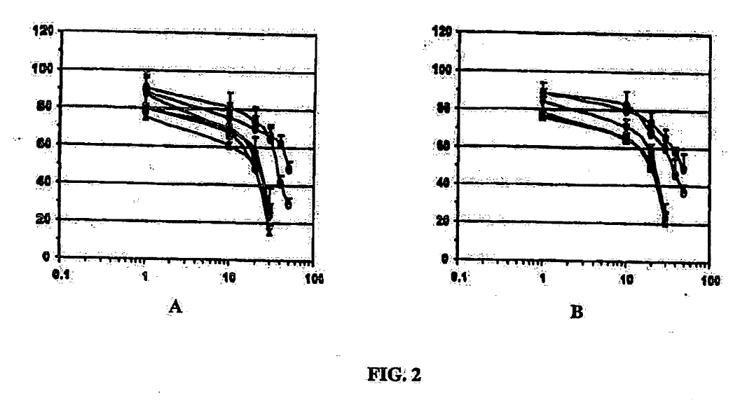

FIG. 3 shows a combination index isobologram which indicates that (−)-epigallocatechin gallate and (−)-epicatechin exhibit a synergistic effect on the growth of HT29 human colon cancer cells. The y-axis represents the Combination Index, the rightmost x-axis represents the concentration of (−)-epicatechin in μg/mL, and the leftmost x-axis represents the concentration of (−)-epigallocatechin gallate in μg/mL. A combination index of 1.1-1.3 is representative of moderate antagonism, a combination index of 0.9-1.1 is representative of an additive effect, a combination index of 0.8-0.9 is representative of slight synergism, a combination index of 0.6-0.8 is representative of moderate synergism, and a combination index of 0.4-0.6 is representative of synergism.

FIG. 4 shows the sequence of oligonucleotide primers used for PCR amplification for the IGFBP-3, IGF-1, MMP-7, MMP-9 and TGF-β2 genes.

FIG. 5 shows the expression levels of IGF-1α, IGF-1β, p-IGF-1R, IGFBP-3, and IGF-1 proteins in the HCT116, Caco2, HT29, SW837 and SW480 human colon cancer cell lines. Total protein extracts were prepared from 70% confluent cultures of the indicated cell lines and equivalent amounts of protein (60 μg/lane) were examined by Western blot analysis using the appropriate antibodies.

FIG. 6 shows the effects of EGCG on activation of the IGF-1R and on levels of IGF-1 and IGFBP-3 proteins (FIGS. 6(a) and 6(b)), and mRNAs (FIG. 6(c)) in SW837 human colon cancer cells. The cells were treated with 20 mg/mL EGCG at 0, 3, 6, 12, 24 and 48 hours, and cell extracts were examined by Western blot analysis using the respective antibodies (FIG. 6(a)), or were examined by semiquantitative RT-PCR analysis using IGFBP-3 or IGF-1 specific primers (FIG. 6(c)). The results obtained from semiquantitative RT-PCR analysis were quantitated by densitometry and are displayed in the right panel of FIG. 6C. The effects of a low dose (10 μg/mL) of EGCG on inhibition of the IGF-1R receptor activation and on levels of IGF-1 and IGFBP-3 proteins are shown in FIG. 6(b).

FIG. 7 shows the effects of EGCG on MMPs-7 and 9 mRNAs (FIG. 7(a)) and on TGF-β2 mRNA (FIG. 7(b)) in SW837 human colon cancer cells. The cells were treated with 20 μg/ML EGCG at 0, 3, 6, 12, 24 and 48 hours, and cell extracts were examined by semiquantitative RT-PCR analysis using MMPs-7 and 9 (FIG. 7(a)), and TGF-β2 (FIG. 7(b)) specific primers. The results obtained from semiquantitative RT-PCR analysis were quantitated by densitometry and are displayed in the right panels of FIG. 7(a) and FIG. 7(b). Amplified PCR products obtained with actin specific primers served as internal controls.

FIG. 8 shows the expression levels of COX-2, HER3 and p-HER3 proteins in various colon cancer cell lines. Total protein extracts were prepared from 70% confluent cultures of the indicated cell lines and equivalent amounts of protein (60 μg/lane) were examined using western blot analysis for COX-2 (FIG. 8(a)), and HER3 and p-HER3 (FIG. 8(b)), using the appropriate antibodies.

FIG. 9 shows the effect of EGCG on activation of the EGFR, HER2, and HER3, and on related-downstream signaling pathways, and on cellular levels of both COX-2 protein (FIG. 9(a)) and mRNA (FIG. 9(b)) in SW837 human colon cancer cells. The cells were treated with 20 μg/mL EGCG at 0, 3, 6, 12, 24 and 48 hours, and cell extracts were examined by Western blot analysis using the respective antibodies (FIG. 9(a)) or were examined by semiquantitative RT-PCR analysis using COX-2 specific primers (FIG. 9(b)). An antibody to actin served as the loading control (FIG. 9(a)). Amplified PCR products obtained with actin specific primers served as internal controls (FIG. 9(b)). The results of mRNA bands were quantified by densitometry and these values were displayed as fold expression (FIG. 9(b)). Similar results were obtained in a repeat experiment. FIG. 9(c) shows the effects of EGCG on induction of apoptosis in SW837 human colon cancer cells. The cells were treated with 20 mg/mL EGCG or 0.1% DMSO for 48 hours, and cell extracts were then examined for DNA fragmentation using the ELISA DNA fragmentation system. The clear bars represent control cells and the dark bars represent treated cells. An asterisk indicates a significant difference (p<0.05) between the control (DMSO-treated) cells and the EGCG-treated cells.

FIG. 10 shows the effects of EGCG on the transcriptional activity of the following promoters in SW837 human colon cancer cells: COX-2 (FIG. 10(a)), AP-1 (FIG. 10(b)), and NF-κB (FIG. 10(c)). Transient transfection reporter assays were performed with the indicated luciferase reporter in the presence of the indicated concentrations of EGCG. Relative luciferase activity was then determined after 24 hours. An asterisk indicates a significant difference (p<0.05) between the control (DMSO-treated) cells and the EGCG-treated cells.

FIG. 11 shows the effects of EGCG on production of PGE2 by SW837 human colon cancer cells. The cells were treated with the indicated concentrations of EGCG for 18 hours in the presence of 20 mM arachadonic acid in serum-free medium. The cell-free medium was then collected and assayed for released PGE2. An asterisk indicates a significant difference (p<0.05) between the control (DMSO-treated) cells and the EGCG-treated cells.

FIG. 12 shows the effects of a low dose of EGCG in SW837 human colon cancer cells. FIG. 12(a) shows the inhibition of growth of SW837 human colon cancer cells. The cells were treated with EGCG (1.0 μg/mL or 20 μg/mL) or 0.1% DMSO for 96 hours, and the number of cells were then counted at the indicated times. FIG. 12(b) illustrates the induction of apoptosis in SW837 human colon cancer cells. The cells were treated with 1.0 μg/mL EGCG or 0.1% DMSO for 96 hours, and cell extracts were examined for DNA fragmentation using the ELISA system. An asterisk indicates a significant difference (p<0.05) between the control (DMSO-treated) cells and the EGCG-treated cells. FIG. 12(c) shows the inhibition of the EGFR, HER2 and HER3 receptor activation in SW837 human colon cancer cells and inhibition of cellular levels of COX-3 and Bcl-x_{L} proteins in SW837 human colon cancer cells. The cells were treated with 1.0 μg/mL EGCG or 0.1% DMSO for 72 hours or 96 hours, and cell extracts were examined using western blot analysis using the respective antibodies. An antibody to actin was used as the loading control. The clear bars represent control cells and the dark bars represent treated cells. An asterisk indicates a significant difference (p<0.05) between the control (DMSO-treated) cells and the EGCG-treated cells.

FIG. 13 shows the effects of EGCG on levels of IGF-1, IGF-2 and IGFBP-3 proteins in HepG2 human liver cancer cells. The cells were treated with 20 mg/mL EGCG at 0, 3, 6, 12, 24 and 48 hours, and cell extracts were examined by Western blot analysis using the respective antibodies, or were examined by semiquantitative RT-PCR analysis using IGFBP-3 or IGF-1 specific primers. The results, set forth in FIG. 13, show that EGCG decreased the production of IGF-1 and IGF-2 and increased the production of IGFBP-3.An asterisk indicates a significant difference (p<0.05) between the control (DMSO-treated) cells and the EGCG-treated cells.

### 5. DETAILED DESCRIPTION OF THE INVENTION

The present invention relates to Synergistic Polyphenol Compounds, compositions thereof and methods for treating and preventing cancer in a subject, the methods comprising administering to a subject an effective amount of a Synergistic Polyphenol Compound or composition thereof.

### 5.1 Definitions

The terms used herein having following meaning:

A “subject” is a mammal, e.g., a human, mouse, rat, guinea pig, dog, cat, horse, cow, pig, or non-human primate, such as a monkey, chimpanzee or baboon. In one embodiment, a monkey is a rhesus. In another embodiment, a subject is a human.

The term “Synergistic Polyphenol Compound” refers to a polyphenol compound or pharmaceutically acceptable salt thereof, wherein the compound demonstrates a synergistic or additive effect with: (a) one or more additional Synergistic Polyphenol Compounds or (b) one or more other anticancer agents, for the treatment or prevention of cancer in a subject. Illustrative Synergistic Polyphenol Compounds include, but are not limited to, a catechin, including but not limited to (−)-epicatechin, (−)-epicatechin gallate, (−)-gallocatechin gallate, (−)-epigallocatechin and (−)-epigallocatechin gallate; a phenolic acid, including but not limited to gallic acid, caffeic acid and ellagic acid; a bioflavanoid, including but not limited to an anthocyanin, apigenin, and quercetin; and a complex polyphenol, including but not limited to, a tannin and a lignan, and any combination thereof. In one embodiment, a Synergistic Polyphenol Compound demonstrates a synergistic effect with: (a) one or more additional Synergistic Polyphenol Compounds and/or (b) one or more other anticancer agents, for the treatment or prevention of cancer in a subject. In one embodiment, a Synergistic Polyphenol Compound demonstrates an additive effect with: (a) one or more additional Synergistic Polyphenol Compounds and/or (b) one or more other anticancer agents, for the treatment or prevention of cancer in a subject.

The term “Additive” when used in connection with the Synergistic Polyphenol Compounds, means that the overall therapeutic effect of a combination of: (a) two or more Synergistic Polyphenol Compounds or (b) one or more Synergistic Polyphenol Compounds and one or more other anticancer agents, when administered as combination therapy for the treatment of cancer, is equal to the sum of the therapeutic effects of these agents when each is adminstered alone as monotherapy.

The term “Synergistic” when used in connection with the Synergistic Polyphenol Compounds, means that the overall therapeutic effect of a combination of: (a) two or more Synergistic Polyphenol Compounds or (b) one or more Synergistic Polyphenol Compounds and one or more other anticancer agents, when administered as combination therapy for the treatment of cancer, is greater than the sum of the therapeutic effects of these agents when each is adminstered alone as monotherapy.

The term “Synergistic Polyphenol Composition” refers to a composition comprising two or more Synergistic Polyphenol Compounds or pharmaceutically acceptable salts thereof, and a physiologically acceptable carrier or vehicle.

The term “in isolated form” as used herein means separated from other components of a reaction mixture or natural source. In certain embodiments, an isolate contains at least 30%, at least 35%, at least 40%, at least 45%, at least 50%, at least 55%, at least 60%, at least 65%, at least 70%, at least 75%, at least 80%, at least 85%, at least 90%, at least 95%, or at least 98% of a Synergistic Polyphenol Compound by weight of the isolate.

The phrase “consisting essentially of” as used herein in connection with the identity of a Synergistic Polyphenol Composition means that the Synergistic Polyphenol Composition contains only those Synergistic Polyphenol Compounds that are set forth as components of the Synergistic Polyphenol Composition. It is to be understood that a Synergistic Polyphenol Composition that consists essentially of specifically named Synergistic Polyphenol Compounds may contain other materials in addition to the named Synergistic Polyphenol Compounds, such that the other materials do not materially affect the basic and novel characteristic(s) of the Synergistic Polyphenol Composition.

The term “Poly E” refers to polyphenon E (Mitsui-Norin, Ltd., Halifax, Canada), which is a standardized, commercially available green tea extract and contains (−)-epigallocatechin gallate, (−)-epigallocatechin, (−)-epicatechin gallate, (−)-epicatechin and (−)-gallocatechin gallate.

The phrase “pharmaceutically acceptable salt,” as used herein, is a salt formed from an acid and a basic nitrogen group of a Synergistic Polyphenol Compound. Illustrative salts include, but are not limited, to sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate, and pamoate (i.e., 1,1′-methylene-bis-(2-OH-3-naphthoate)) salts. The term “pharmaceutically acceptable salt” also refers to a salt prepared from a Synergistic Polyphenol Compound having an acidic functional group, such as a carboxylic acid functional group, and a pharmaceutically acceptable inorganic or organic base. Suitable bases include, but are not limited to, hydroxides of alkali metals such as sodium, potassium, and lithium; hydroxides of alkaline earth metal such as calcium and magnesium; hydroxides of other metals, such as aluminum and zinc; ammonia, and organic amines, such as unsubstituted or hydroxy-substituted mono-, di-, or tri-alkylamines, dicyclohexylamine; tributyl amine; pyridine; N-methyl, N-ethylamine; diethylamine; triethylamine; mono-, bis-, or tris-(2-hydroxy substituted lower alkylamines), such as mono-; bis-, or tris-(2-hydroxyethyl)amine, 2-hydroxy-tert-butylamine, or tris-(hydroxymethyl)methylamine, N,N-di-lower alkyl-N-(hydroxy lower alkyl)-amines, such as N,N-dimethyl-N-(2-hydroxyethyl)amine or tri-(2-hydroxyethyl)amine; N-methyl-D-glucamine; and amino acids such as arginine, lysine, and the like. The term “pharmaceutically acceptable salt” also includes a hydrate of a Synergistic Polyphenol Compound.

The following abbreviations are used herein and have the following meanings: COX-2 is cyclooxygenase 2; DMSO is N,N-dimethylsulfoxide; EGCG is epigallocatechin gallate; EGFR is epidermal growth factor receptor; ERK is extracellular related kinase; HER2 is herceptin 2; HER3 is herceptin 3; IGF-1 is insulin-like growth factor 1; IGF-1 R is insulin-like growth factor 1 receptor; IGFBP-3 is insulin-like growth factor binding protein 3; PGE₂ is prostaglandin E₂; PBS is phosphate-buffered saline; RTK is receptor tyrosine kinase; and RT-PCR is reverse transcriptase polymerase chain reaction.

### 5.2 The Synergistic Polyphenol Compounds

As stated above, the present invention encompasses methods for treating or preventing cancer in a subject, the methods comprising administering to the subject: (i) an effective amount of a Synergistic Polyphenol Composition or (ii) two or more Synergistic Polyphenol Compounds wherein the amounts of the two or more Synergistic Polyphenol Compounds are together synergistically effective to treat or prevent cancer.

Illustrative Synergistic Polyphenol Compounds useful in the Synergistic Polyphenol Compositions and present methods for treating or preventing cancer, include, but are not limited to the following compounds and pharmaceutically acceptable salts thereof: a catechin, including but not limited to (−)-epicatechin, (−)-epicatechin gallate, (−)-gallocatechin gallate, (−)-epigallocatechin and (−)-epigallocatechin gallate; a phenolic acid, including but not limited to gallic acid, caffeic acid and ellagic acid; a bioflavanoid, including but not limited to an anthocyanin, apigenin, and quercetin; and a complex polyphenol, including but not limited to, a tannin and a lignan, and any combination thereof.

In one embodiment, the Synergistic Polyphenol Compound is (−)-epigallocatechin gallate.

In another embodiment, the Synergistic Polyphenol Compound is (+)-polyphenol.

In another embodiment, the Synergistic Polyphenol Compound is (−)-epicatechin.

In still another embodiment, the Synergistic Polyphenol Compound is (−)-epicatechin gallate.

In yet another embodiment, the Synergistic Polyphenol Compound is (−)-gallocatechin gallate.

In a further embodiment, the Synergistic Polyphenol Compound is (−)-epigallocatechin.

In one embodiment, the Synergistic Polyphenol Compound is a bioflavanoid.

In another embodiment, the Synergistic Polyphenol Compound is a phenolic acid.

In another embodiment, the Synergistic Polyphenol Compound is a tannin.

In still another embodiment, the Synergistic Polyphenol Compound is a lignan.

The Synergistic Polyphenol Compounds may be purchased from commercial sources (e.g., Sigma Chemical, St. Louis, Mo.), prepared synthetically using methods well-known to one skilled in the art of synthetic organic chemistry, or extracted from natural sources using methods well-known to one skilled in the arts of chemistry and/or biology and/or related arts.

It is possible for some of the Synergistic Polyphenol Compounds to have one or more chiral centers and as such these Synergistic Polyphenol Compounds can exist in various stereoisomeric forms. Accordingly, the present invention is understood to encompass all possible stereoisomers.

In one embodiment, a Synergistic Polyphenol Compound is obtained from a natural product extract. In a specific embodiment, a Synergistic Polyphenol Compound is obtained from a green tea extract.

In another embodiment, a Synergistic Polyphenol Compound is in isolated form.

### 5.3 The Synergistic Polyphenol Compositions

In one embodiment, the Synergistic Polyphenol Compositions comprise two or more Synergistic Polyphenol Compounds and a physiologically acceptable carrier or vehicle, and are useful for treating or preventing cancer in a subject.

In one embodiment, a Synergistic Polyphenol Composition comprises (−)-epigallocatechin gallate.

In another embodiment, a Synergistic Polyphenol Composition comprises (+)-polyphenol.

In another embodiment, a Synergistic Polyphenol Composition comprises (−)-epicatechin.

In still another embodiment, a Synergistic Polyphenol Composition comprises (−)-epicatechin gallate.

In yet another embodiment, a Synergistic Polyphenol Composition comprises (−)-gallocatechin gallate.

In a further embodiment, a Synergistic Polyphenol Composition comprises (−)-epigallocatechin.

In one embodiment, a Synergistic Polyphenol Composition comprises a bioflavanoid.

In another embodiment, a Synergistic Polyphenol Composition comprises a phenolic acid.

In another embodiment, a Synergistic Polyphenol Composition comprises a tannin.

In still another embodiment, a Synergistic Polyphenol Composition comprises a lignan.

In one embodiment, a Synergistic Polyphenol Composition comprises (−)-epigallocatechin gallate and (−)-epicatechin.

In another embodiment, a Synergistic Polyphenol Composition comprises (−)-epigallocatechin gallate and (+)-polyphenol.

In another embodiment, a Synergistic Polyphenol Composition comprises (−)-epigallocatechin gallate and (−)-epicatechin gallate.

In still another embodiment, a Synergistic Polyphenol Composition comprises (−)-epigallocatechin gallate and (−)-gallocatechin gallate.

In a further embodiment, a Synergistic Polyphenol Composition comprises (−)-epigallocatechin gallate and (−)-epigallocatechin.

In one embodiment, a Synergistic Polyphenol Composition comprises (−)-epigallocatechin gallate, (−)-epicatechin, (−)-epicatechin gallate, (−)-gallocatechin gallate and (−)-epigallocatechin.

In one embodiment, a Synergistic Polyphenol Composition comprises (−)-epigallocatechin gallate and epicatechin such that the ratio of (−)-epigallocatechin gallate to (−)-epicatechin by weight is from (a) about 20 to about 1 to 10 to (b) about 1 to about 10.

In various embodiments, a Synergistic Polyphenol Composition comprises (−)-epigallocatechin gallate and epicatechin such that the ratio of (−)-epigallocatechin gallate to (−)-epicatechin by weight is about 20 to about 1; about 10 to about 1; about 2 to about 1; about 1 to about 1; about 2 to about 5, about 1 to about 5, or about 1 to about 10.

In one embodiment, the Synergistic Polyphenol Compositions consist essentially of two or more Synergistic Polyphenol Compounds and a physiologically acceptable carrier or vehicle, and are useful for treating or preventing cancer in a subject.

In one embodiment, a Synergistic Polyphenol Composition consists essentially of (−)-epigallocatechin gallate and (−)-epicatechin.

In another embodiment, a Synergistic Polyphenol Composition consists essentially of (−)-epigallocatechin gallate and (+)-polyphenol.

In another embodiment, a Synergistic Polyphenol Composition consists essentially of (−)-epigallocatechin gallate and (−)-epicatechin gallate.

In still another embodiment, a Synergistic Polyphenol Composition consists essentially of (−)-epigallocatechin gallate and (−)-gallocatechin gallate.

In a further embodiment, a Synergistic Polyphenol Composition consists essentially of (−)-epigallocatechin gallate and (−)-epigallocatechin.

In one embodiment, a Synergistic Polyphenol Composition consists essentially of (−)-epigallocatechin gallate, (−)-epicatechin, (−)-epicatechin gallate, (−)-gallocatechin gallate and (−)-epigallocatechin.

In one embodiment, a Synergistic Polyphenol Composition consists essentially of (−)-epigallocatechin gallate and epicatechin such that the ratio of (−)-epigallocatechin gallate to (−)-epicatechin by weight is from (a) about 20 to about 1 to 10 to (b) about 1 to about 10.

In various embodiments, a Synergistic Polyphenol Composition consists essentially of (−)-epigallocatechin gallate and (−)-epicatechin such that the ratio of (−)-epigallocatechin gallate to (−)-epicatechin by weight is about 20 to about 1; about 10 to about 1; about 2 to about 1; about 1 to about 1; about 2 to about 5, about 1 to about 5, or about 1 to about 10.

In one embodiment, one or more of the Synergistic Polyphenol Compounds of a Synergistic Polyphenol Composition is in isolated form.

### 5.3.1 Obtaining the Synergistic Polyphenol Compositions

The Synergistic Polyphenol Compositions can be prepared using combinations of various isolated or crude Synergistic Polyphenol Compounds or may be obtained as extracts from natural products, such as green tea.

### 5.3.1.1 Green Tea Extracts

Green tea leaves have been found to contain numerous biologically active compounds that are potentially useful in medical and veterinary applications, including various members of the polyphenol family of natural products. As such, in one embodiment, a Synergistic Polyphenol Composition can be obtained from green tea leaves using extraction procedures well known to one skilled in the relevant art. The extraction procedures may be carried out using water, polar organic solvents, non-polar organic solvents, supercritical fluids, or mixtures thereof. Organic solvents useful for extracting a Synergistic Polyphenol Composition from green tea leaves include, but are not limited to alcohols, such as methanol, ethanol, n-propanol, isopropanol, n-butanol, isobutanol, sec-butanol and tert-butanol; ketones, such as acetone, methyl ethyl ketone, and ethyl acetate; ethers, such as diethyl ether, diphenyl ether, tetrahydrofuran, and dioxane; aliphatic hydrocarbons, such as pentanes, hexanes, and heptanes; aromatic hydrocarbons such as benzene, toluene, naphthalene, and xylenes; alkyl halides, such as carbon tetrachloride, choroform and methylene chloride; amides, such as dimethylformamide and hexamethylphosporamide; carboxylic acids, such as formic acid and acetic acid; and sulfoxides, such as dimethylsulfoxide.

As stated above, the present invention encompasses methods for treating or preventing cancer in a subject, the methods comprising administering to the subject an effective amount of a Synergistic Polyphenol Composition extracted from green tea leaves. Green tea extracts containing a Synergistic Polyphenol Composition include both oil and water soluble extracts and can be obtained from commercial sources (e.g., Nature's Resource, Mission Hills, Calif., or Herbasin, Beijing, China) or can be obtained directly from green tea leaves using known extraction methods, such as those disclosed in European Patent No. EP 1402869 to Schneider, which is hereby incorporated by reference in its entirety.

In one embodiment, the Synergistic Polyphenol Composition is Poly E.

In another embodiment, the Synergistic Polyphenol Composition is extracted from a natural source.

### 5.4 Uses of the Compounds and Compositions of the Invention

### 5.4.1 Treatment or Prevention of Cancer

The Synergistic Polyphenol Compounds or Synergistic Polyphenol Compositions are useful for treating or preventing cancer.

In one embodiment, the invention provides a method for treating cancer in a subject, the method comprising:

(a) administering to the subject a first Synergistic Polyphenol Compound; and

(b) administering to the subject a second Synergistic Polyphenol Compound, wherein the first and second compounds are different and wherein the amounts of the first and second compounds administered are together synergistically effective to treat or prevent cancer.

In another embodiment, the invention provides a method for treating colorectal cancer or liver cancer in a subject, the method comprising:

(a) administering to the subject a first Synergistic Polyphenol Compound; and

(b) administering to the subject a second Synergistic Polyphenol Compound, wherein the first and second compounds are different and wherein the amounts of the first and second compounds administered are together synergistically effective to treat or prevent colorectal cancer or liver cancer.

In one embodiment, the cancer being treated or prevented is colorectal cancer.

In another embodiment, the cancer being treated or prevented is liver cancer.

In one embodiment, the first compound is (−)-epigallocatechin gallate.

In another embodiment the first compound is (−)-epicatechin.

In another embodiment, the first compound is (−)-epigallocatechin gallate and the second compound is (−)-epicatechin.

In still another embodiment, the first compound is (−)-epigallocatechin gallate and the second compound is (−)-epicatechin, wherein the ratio of the amount of (−)-epicatechin administered to the amount of (−)-epigallocatechin gallate administered to is about 5 to about 2.

In a further embodiment, the first compound is (−)-epigallocatechin gallate and the second compound is (−)-epicatechin, wherein the ratio of the amount of (−)-epicatechin administered to the amount of (−)-epigallocatechin gallate administered to is about 5 to about 1.

In various embodiments, the first compound is (−)-epigallocatechin gallate and the second compound is (−)-epicatechin such that the ratio of (−)-epigallocatechin gallate administered to the amount of (−)-epicatechin administered is about 20 to about 1; about 10 to about 1; about 2 to about 1; about 1 to about 1; about 2 to about 5, about 1 to about 5, or about 1 to about 10.

In another embodiment, the invention provides a method for treating cancer in a subject, the method comprising administering to the subject an effective amount of a Synergistic Polyphenol Composition.

Examples of cancers treatable or preventable using the Synergistic Polyphenol Compounds or Synergistic Polyphenol Compositions include, but are not limited to, the cancers disclosed below in Table 1 and metastases thereof.

TABLE 1

Solid tumors, including but not limited to:
fibrosarcoma
myxosarcoma
liposarcoma
chondrosarcoma
osteogenic sarcoma
chordoma
angiosarcoma
endotheliosarcoma
lymphangiosarcoma
lymphangioendotheliosarcoma
synovioma
mesothelioma
Ewing's tumor
leiomyosarcoma
rhabdomyosarcoma
colon cancer
colorectal cancer
kidney cancer
pancreatic cancer
bone cancer
breast cancer
ovarian cancer
prostate cancer
esophageal cancer
stomach cancer
oral cancer
nasal cancer
throat cancer
squamous cell carcinoma
basal cell carcinoma
adenocarcinoma
sweat gland carcinoma
sebaceous gland carcinoma
papillary carcinoma
papillary adenocarcinomas
cystadenocarcinoma
medullary carcinoma
bronchogenic carcinoma
renal cell carcinoma
hepatoma
liver cancer
bile duct carcinoma
choriocarcinoma
seminoma
embryonal carcinoma
Wilms' tumor
cervical cancer
uterine cancer
testicular cancer
small cell lung carcinoma
bladder carcinoma
lung cancer
epithelial carcinoma
skin cancer
melanoma
neuroblastoma
retinoblastoma
blood-borne cancers, including but not limited to:
acute lymphoblastic leukemia (“ALL”)
acute lymphoblastic B-cell leukemia
acute lymphoblastic T-cell leukemia
acute myeloblastic leukemia (“AML”)
acute promyelocytic leukemia (“APL”)
acute monoblastic leukemia
acute erythroleukemic leukemia
acute megakaryoblastic leukemia
acute myelomonocytic leukemia
acute nonlymphocyctic leukemia
acute undifferentiated leukemia
chronic myelocytic leukemia (“CML”)
chronic lymphocytic leukemia (“CLL”)
hairy cell leukemia
multiple myeloma
acute and chronic leukemias:
lymphoblastic
myelogenous
lymphocytic
myelocytic leukemias
Lymphomas:
Hodgkin's disease
non-Hodgkin's Lymphoma
Multiple myeloma
Waldenström's macroglobulinemia
Heavy chain disease
Polycythemia vera
CNS and brain cancers:
glioma
pilocytic astrocytoma
astrocytoma
anaplastic astrocytoma
glioblastoma multiforme
medulloblastoma
craniopharyngioma
ependymoma
pinealoma
hemangioblastoma
acoustic neuroma
oligodendroglioma
meningioma
vestibular schwannoma
adenoma
metastatic brain tumor
meningioma
spinal tumor
medulloblastoma

In one embodiment the cancer is lung cancer, breast cancer, colorectal cancer, prostate cancer, a leukemia, a lymphoma, a non-Hodgkin's lymphoma, a skin cancer, a brain cancer, a cancer of the central nervous system, ovarian cancer, uterine cancer, stomach cancer, pancreatic cancer, esophageal cancer, kidney cancer, liver cancer, or a head and neck cancer.

In one embodiment, the cancer is a solid tumor.

In another embodiment, the cancer is a systemic cancer, such as a leukemia or a lymphoma.

In a specific embodiment, the cancer is colorectal cancer.

In another specific embodiment the cancer is breast cancer.

In another specific embodiment the cancer is liver cancer.

In one embodiment, the subject has previously undergone or is presently undergoing treatment for cancer. Such previous treatments include, but are not limited to, prior chemotherapy, radiation therapy, surgery, or immunotherapy, such as a cancer vaccine.

The Synergistic Polyphenol Compounds or Synergistic Polyphenol Compositions are also useful for the treatment or prevention of a cancer caused by a virus. Such viruses include human papilloma virus, which can lead to cervical cancer (see, e.g., Hernandez-Avila et al., Archives of Medical Research (1997) 28:265-271); Epstein-Barr virus (EBV), which can lead to lymphoma (see, e.g., Herrmann et al., J Pathol (2003) 199(2):140-5); hepatitis B or C virus, which can lead to liver carcinoma (see, e.g., E1-Serag, J Clin Gastroenterol (2002) 35(5 Suppl 2):S72-8); human T cell leukemia virus (HTLV)-I, which can lead to T-cell leukemia (see e.g., Mortreux et al., Leukemia (2003) 17(1):26-38); human herpesvirus-8 infection, which can lead to Kaposi's sarcoma (see, e.g., Kadow et al., Curr Opin Investig Drugs (2002) 3(11):1574-9); and Human Immune deficiency Virus (HIV) infection, which can lead to cancer as a consequence of immunodeficiency (see, e.g., Dal Maso et al., Lancet Oncol (2003) 4(2):110-9).

The Synergistic Polyphenol Compounds or Synergistic Polyphenol Compositions can also be administered to prevent the progression of a cancer, including but not limited to the cancers listed in Table 1. Such prophylactic use includes that in which non-neoplastic cell growth consisting of hyperplasia, metaplasia, or most particularly, dysplasia has occurred.

Alternatively or in addition to the presence of abnormal cell growth characterized as hyperplasia, metaplasia, or dysplasia, the presence of one or more characteristics of a transformed phenotype, or of a malignant phenotype, displayed in vivo or displayed in vitro by a cell sample from an animal, can indicate the desirability of prophylactic/therapeutic administration of the Synergistic Polyphenol Compounds or Synergistic Polyphenol Compositions. Such characteristics of a transformed phenotype include morphology changes, looser substratum attachment, loss of contact inhibition, loss of anchorage dependence, protease release, increased sugar transport, decreased serum requirement, expression of fetal antigens, disappearance of the 250,000 dalton cell surface protein, etc. (see also id., at pp. 84-90 for characteristics associated with a transformed or malignant phenotype).

In a specific embodiment, leukoplakia, a benign-appearing hyperplastic or dysplastic lesion of the epithelium, or Bowen's disease, a carcinoma in situ, are treatable or preventable according to the present methods.

In another embodiment, fibrocystic disease (cystic hyperplasia, mammary dysplasia, particularly adenosis (benign epithelial hyperplasia)) are treatable or preventable according to the present methods.

In other embodiments, a subject that exhibits one or more of the following predisposing factors for malignancy can be administered an amount of two or more Synergistic Polyphenol Compounds or an amount of a Synergistic Polyphenol Composition which is effective to treat or prevent cancer: a chromosomal translocation associated with a malignancy (e.g., the Philadelphia chromosome for chronic myelogenous leukemia, t(14;18) for follicular lymphoma); familial polyposis or Gardner's syndrome; benign monoclonal gammopathy; a first degree kinship with persons having a cancer or precancerous disease showing a Mendelian (genetic) inheritance pattern (e.g., familial polyposis of the colorectal, Gardner's syndrome, hereditary exostosis, polyendocrine adenomatosis, medullary thyroid carcinoma with amyloid production and pheochromocytoma, Peutz-Jeghers syndrome, neurofibromatosis of Von Recklinghausen, retinoblastoma, carotid body tumor, cutaneous melanocarcinoma, intraocular melanocarcinoma, xeroderma pigmentosum, ataxia telangiectasia, Chediak-Higashi syndrome, albinism, Fanconi's aplastic anemia, and Bloom's syndrome; and exposure to carcinogens (e.g., smoking, second-hand smoke exposure, and inhalation of or contacting with certain chemicals).

### 5.4.2 Combination Chemotherapy for the Treatment of Cancer

In one embodiment, the present methods for treating cancer or preventing cancer further comprise administering another anticancer agent that is not a Synergistic Polyphenol Compound.

In one embodiment, the present invention provides methods for treating or preventing cancer in a subject, the method comprising the administration of an effective amount of: (i) a Synergistic Polyphenol Composition or two or more Synergistic Polyphenol Compounds, and (ii) another anticancer agent.

In one embodiment, (i) a Synergistic Polyphenol Composition or two or more Synergistic Polyphenol Compounds, and (ii) another anticancer agent are administered in doses commonly employed when such agents are used alone for the treatment of cancer.

In another embodiment, (i) a Synergistic Polyphenol Composition or two or more Synergistic Polyphenol Compounds, and (ii) another anticancer agent act synergistically and are administered in doses that are less than the doses commonly employed when such agents are used alone for the treatment of cancer.

The dosage of the (i) a Synergistic Polyphenol Composition or two or more Synergistic Polyphenol Compounds, and (ii) another anticancer agent administered as well as the dosing schedule can depend on various parameters, including, but not limited to, the cancer being treated, the subject's general health, and the administering physician's discretion.

A Synergistic Polyphenol Composition or Synergistic Polyphenol Compound can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concurrently with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of the other anticancer agent to a subject in need thereof. In various embodiments, i) a Synergistic Polyphenol Composition or two or more Synergistic Polyphenol Compounds, and (ii) another anticancer agent are administered 1 minuteute apart, 10 minutes apart, 30 minutes apart, less than 1 hour apart, 1 hour to 2 hours apart, 2 hours to 3 hours apart, 3 hours to 4 hours apart, 4 hours to 5 hours apart, 5 hours to 6 hours apart, 6 hours to 7 hours apart, 7 hours to 8 hours apart, 8 hours to 9 hours apart, 9 hours to 10 hours apart, 10 hours to 11 hours apart, 11 hours to 12 hours apart, no more than 24 hours apart, or no more than 48 hours apart. In one embodiment, i) a Synergistic Polyphenol Composition and (ii) another anticancer agent are administered with 3 hours. In another embodiment, i) a Synergistic Polyphenol Composition and (ii) another anticancer agent are administered 1 minuteute to 24 hours apart.

In one embodiment, a Synergistic Polyphenol Composition further comprises an effective amount of another anticancer agent, such that the Synergistic Polyphenol Compounds of the Synergistic Polyphenol Composition and the other anticancer agent are present in the same composition. In one embodiment, this composition is useful for oral administration. In another embodiment, this composition is useful for intravenous administration.

Cancers that can be treated or prevented by administering and effective amount of (i) a Synergistic Polyphenol Composition or two or more Synergistic Polyphenol Compounds, and (ii) another anticancer agent include, but are not limited to, the list of cancers set forth in Table 1.

In one embodiment the cancer is lung cancer, breast cancer, colorectal cancer, prostate cancer, a leukemia, a lymphoma, a non-Hodgkin's lymphoma, a skin cancer, a brain cancer, a cancer of the central nervous system, ovarian cancer, uterine cancer, stomach cancer, pancreatic cancer, esophageal cancer, kidney cancer, liver cancer, or a head and neck cancer.

In another embodiment, the cancer is colorectal cancer.

In still another embodiment the cancer is breast cancer.

In another embodiment the cancer is liver cancer.

The (i) Synergistic Polyphenol Composition or two or more Synergistic Polyphenol Compounds, and (ii) other anticancer agent, can act additively or synergistically. A synergistic combination of the (i) Synergistic Polyphenol Composition or two or more Synergistic Polyphenol Compounds, and (ii) other anticancer agent might allow the use of lower dosages of the (i) Synergistic Polyphenol Composition or two or more Synergistic Polyphenol Compounds, and/or (ii) other anticancer agent and/or less frequent dosages of the (i) Synergistic Polyphenol Composition or two or more Synergistic Polyphenol Compounds, and (ii) other anticancer agent, and/or to administer the (i) Synergistic Polyphenol Composition or two or more Synergistic Polyphenol Compounds, and/or (ii) other anticancer agent less frequently can reduce any toxicity associated with the administration of (i) and/or (ii) to a subject without reducing the efficacy of (i) and/or (ii) in the treatment of cancer. In addition, a synergistic effect might result in the improved efficacy of these agents in the treatment of cancer and/or the reduction of any adverse or unwanted side effects associated with the use of either agent alone.

In one embodiment, a (i) Synergistic Polyphenol Composition or a Synergistic Polyphenol Compound, and (ii) another anticancer agent act synergistically when administered in doses typically employed when such agents are used alone for the treatment of cancer. In another embodiment, a (i) Synergistic Polyphenol Composition or a Synergistic Polyphenol Compound, and (ii) another anticancer agent, act synergistically when administered in doses that are less than doses typically employed when such agents are used alone for the treatment of cancer.

In one embodiment, the administration of an effective amount of a (i) Synergistic Polyphenol Composition or a Synergistic Polyphenol Compound, and (ii) another anticancer agent inhibits the resistance of a cancer to the other anticancer agent.

Suitable other anticancer agents useful in the methods and compositions of the present invention include, but are not limited to temozolomide, a topoisomerase I inhibitor, procarbazine, dacarbazine, gemcitabine, capecitabine, methotrexate, taxol, taxotere, mercaptopurine, thioguanine, hydroxyurea, cytarabine, cyclophosphamide, ifosfamide, nitrosoureas, cisplatin, carboplatin, mitomycin, dacarbazine, procarbizine, etoposide, teniposide, campathecins, bleomycin, doxorubicin, idarubicin, daunorubicin, dactinomycin, plicamycin, mitoxantrone, L-asparaginase, doxorubicin, epirubicin, 5-fluorouracil, taxanes such as docetaxel and paclitaxel, leucovorin, levamisole, irinotecan, estramustine, etoposide, nitrogen mustards, BCNU, nitrosoureas such as carmustine and lomustine, vinca alkaloids such as vinblastine, vincristine and vinorelbine, platinum complexes such as cisplatin, carboplatin and oxaliplatin, imatinib mesylate, hexamethylmelamine, topotecan, tyrosine kinase inhibitors, tyrphostins herbimycin A, genistein, erbstatin, and lavendustin A.

In one embodiment, the other anticancer agents useful in the methods and compositions of the present invention include, but are not limited to, a drug listed in Table 2 or a pharmaceutically acceptable salt thereof.

TABLE 2

| | |
|---|---|
| Alkylating agents | |
| Nitrogen mustards: | Cyclophosphamide |
| | Ifosfamide |
| | Trofosfamide |
| | Chlorambucil |
| Nitrosoureas: | Carmustine (BCNU) |
| | Lomustine (CCNU) |
| Alkylsulphonates: | Busulfan |
| | Treosulfan |
| Triazenes: | Dacarbazine |
| | Procarbazine |
| | Temozolomide |
| Platinum containing complexes: | Cisplatin |
| | Carboplatin |
| | Aroplatin |
| | Oxaliplatin |
| Plant Alkaloids | |
| Vinca alkaloids: | Vincristine |
| | Vinblastine |
| | Vindesine |
| | Vinorelbine |
| Taxoids: | Paclitaxel |
| | Docetaxel |
| DNA Topoisomerase Inhibitors | |
| Epipodophyllins: | Etoposide |
| | Teniposide |
| | Topotecan |
| | Irinotecan |
| | 9-aminocamptothecin |
| | Camptothecin |
| | Crisnatol |
| Mitomycins: | Mitomycin C |
| | Anti-metabolites |
| Anti-folates: | |
| DHFR inhibitors: | Methotrexate |
| | Trimetrexate |
| IMP dehydrogenase Inhibitors: | Mycophenolic acid |
| | Tiazofurin |
| | Ribavirin |
| | EICAR |
| Ribonuclotide reductase | Hydroxyurea |
| Inhibitors: | Deferoxamine |
| Pyrimidine analogs: | |
| Uracil analogs: | 5-Fluorouracil |
| | Fluoxuridine |
| | Doxifluridine |
| | Ralitrexed |
| Cytosine analogs: | Cytarabine (ara C) |
| | Cytosine arabinoside |
| | Fludarabine |
| | Gemcitabine |
| | Capecitabine |
| Purine analogs: | Mercaptopurine |
| | Thioguanine |
| | O-6-benzylguanine |
| DNA Antimetabolites: | 3-HP |
| | 2′-deoxy-5-fluorouridine |
| | 5-HP |
| | alpha-TGDR |
| | aphidicolin glycinate |
| | ara-C |
| | 5-aza-2′-deoxycytidine |
| | beta-TGDR |
| | cyclocytidine |
| | guanazole |
| | inosine glycodialdehyde |
| | macebecin II |
| | Pyrazoloimidazole |
| Hormonal therapies: | |
| Receptor antagonists: | |
| Anti-estrogen: | Tamoxifen |
| | Raloxifene |
| | Megestrol |
| LHRH agonists: | Goscrclin |
| | Leuprolide acetate |
| Anti-androgens: | Flutamide |
| | Bicalutamide |
| Retinoids/Deltoids | Cis-retinoic acid |
| Vitamin A derivative: | All-trans retinoic acid (ATRA-IV) |
| Vitamin D3 analogs: | EB 1089 |
| | CB 1093 |
| | KH 1060 |
| Photodynamic therapies: | Vertoporfin (BPD-MA) |
| | Phthalocyanine |
| | Photosensitizer Pc4 |
| | Demethoxy-hypocrellin A |
| | (2BA-2-DMHA) |
| Cytokines: | Interferon-α |
| | Interferon-β |
| | Interferon-γ |
| | Tumor necrosis factor |
| | Interleukin-2 |
| Angiogenesis Inhibitors: | Angiostatin (plasminogen |
| | fragment) |
| | antiangiogenic antithrombin III |
| | Angiozyme |
| | ABT-627 |
| | Bay 12-9566 |
| | Benefin |
| | Bevacizumab |
| | BMS-275291 |
| | cartilage-derived inhibitor (CDI) |
| | CAI |
| | CD59 complement fragment |
| | CEP-7055 |
| | Col 3 |
| | Combretastatin A-4 |
| | Endostatin (collagen XVIII |
| | fragment) |
| | Fibronectin fragment |
| | Gro-beta |
| | Halofuginone |
| | Heparinases |
| | Heparin hexasaccharide fragment |
| | HMV833 |
| | Human chorionic gonadotropin |
| | (hCG) |
| | IM-862 |
| | Interferon alpha/beta/gamma |
| | Interferon inducible protein (IP- |
| | 10) |
| | Interleukin-12 |
| | Kringle 5 (plasminogen fragment) |
| | Marimastat |
| | Metalloproteinase inhibitors |
| | (TIMPs) |
| | 2-Methoxyestradiol |
| | MMI 270 (CGS 27023A) |
| | MoAb IMC-1C11 |
| | Neovastat |
| | NM-3 |
| | Panzem |
| | PI-88 |
| | Placental ribonuclease inhibitor |
| | Plasminogen activator inhibitor |
| | Platelet factor-4 (PF4) |
| | Prinomastat |
| | Prolactin 16 kD fragment |
| | Proliferin-related protein (PRP) |
| | PTK 787/ZK 222594 |
| | Retinoids |
| | Solimastat |
| | Squalamine |
| | SS 3304 |
| | SU 5416 |
| | SU6668 |
| | SU11248 |
| | Tetrahydrocortisol-S |
| | Tetrathiomolybdate |
| | Thalidomide |
| | Thrombospondin-1 (TSP-1) |
| | TNP-470 |
| | Transforming growth factor-beta |
| | (TGF-b) |
| | Vasculostatin |
| | Vasostatin (calreticulin fragment) |
| | ZD6126 |
| | ZD 6474 |
| | farnesyl transferase inhibitors |
| | (FTI) |
| | Bisphosphonates |
| Antimitotic agents: | Allocolchicine |
| | Halichondrin B |
| | Colchicine |
| | colchicine derivative |
| | dolstatin 10 |
| | Maytansine |
| | Rhizoxin |
| | Thiocolchicine |
| | trityl cysteine |
| Others: | |
| Protein Kinase G inhibitors: | OSI 461 |
| | Exisulind |
| Tyrosine Kinase inhibitors: | Iressa |
| | Tarceva |
| Dopaminergic neurotoxins: | 1-methyl-4-phenylpyridinium ion |
| Cell cycle inhibitors: | Staurosporine |
| Actinomycins: | Actinomycin D |
| | Dactinomycin |
| Bleomycins: | Bleomycin A2 |
| | Bleomycin B2 |
| | Peplomycin |
| Anthracyclines: | Daunorubicin |
| | Doxorubicin |
| | Idarubicin |
| | Epirubicin |
| | Pirarubicin |
| | Zorubicin |
| | Mitoxantrone |
| MDR inhibitors: | Verapamil |
| Ca²⁺ ATPase inhibitors: | Thapsigargin |

In one embodiment, the other anticancer agent is OSI 461.

In another embodiment, the other anticancer agent is Iressa.

In still another embodiment, the other anticancer agent is taxol.

In a further embodiment, the other anticancer agent is 5-fluorouracil.

In yet another embodiment, the other anticancer agent is a platinum-based anticancer agent.

In one embodiment, the platinum-based anticancer agent is cisplatin, carboplatin or oxaliplatin.

### 5.4.3 Multi-Therapy for Cancer

A Synergistic Polyphenol Compound or Synergistic Polyphenol Composition can be administered to a subject that has undergone or is currently undergoing one or more additional anticancer therapies including, but not limited to, surgery, radiation therapy, or immunotherapy, such as cancer vaccines.

In one embodiment, the invention provides methods for treating or preventing cancer comprising administering to a subject in need thereof (a) an effective amount of a Synergistic Polyphenol Composition or two or more Synergistic Polyphenol Compounds, and (b) another anticancer therapy including, but not limited to, surgery, radiation therapy, or immunotherapy, such as a cancer vaccine.

In one embodiment, the other anticancer therapy is radiation therapy.

In another embodiment, the other anticancer therapy is surgery.

In still another embodiment, the other anticancer therapy is immunotherapy.

In a specific embodiment, the present methods for treating or preventing cancer comprise administering (i) a Synergistic Polyphenol Composition or two or more Synergistic Polyphenol Compounds, and (ii) radiation therapy. The radiation therapy can be administered concurrently with, prior to, or subsequent to the Synergistic Polyphenol Composition or two or more Synergistic Polyphenol Compounds. In various embodiments, radiation therapy is administered at least 30 minutes, one hour, five hours, 12 hours, one day, one week, one month, or several months (e.g., up to three months), prior or subsequent to administration of a Synergistic Polyphenol Composition or Synergistic Polyphenol Compound.

Where the other anticancer therapy is radiation therapy, any radiation therapy protocol can be used depending upon the type of cancer to be treated. For example, but not by way of limitation, X-ray radiation can be administered; in particular, high-energy megavoltage (radiation of greater that 1 MeV energy) can be used for deep tumors, and electron beam and orthovoltage X-ray radiation can be used for skin cancers. Gamma-ray emitting radioisotopes, such as radioactive isotopes of radium, cobalt and other elements, can also be administered.

Additionally, in one embodiment the invention provides methods of treatment of cancer using a Synergistic Polyphenol Composition or two or more Synergistic Polyphenol Compounds as an alternative to chemotherapy or radiation therapy where the chemotherapy or the radiation therapy results in negative side effects, in the subject being treated. The subject being treated can, optionally, be treated with another anticancer therapy such as surgery, radiation therapy, or immunotherapy.

The Synergistic Polyphenol Compositions and Synergistic Polyphenol Compounds can also be used in vitro or ex vivo, such as for the treatment of certain cancers, including, but not limited to leukemias and lymphomas, such treatment involving autologous stem cell transplants. This can involve a process in which the subject's autologous hematopoietic stem cells are harvested and purged of all cancer cells, the subject's remaining bone-marrow cell population is then eradicated via the administration of (i) a Synergistic Polyphenol Composition or two or more Synergistic Polyphenol Compounds, and/or (ii) radiation, and the resultant stem cells are then infused back into the subject. Supportive care can be subsequently provided while bone marrow function is restored and the subject recovers.

### 5.5 Therapeutic/Prophylactic Administration and Compositions of the Invention

Due to their activity, the Synergistic Polyphenol Compounds or Synergistic Polyphenol Compositions are advantageously useful in veterinary and human medicine. As described above, the Synergistic Polyphenol Compounds or Synergistic Polyphenol Compositions are useful for treating or preventing cancer in a subject in need thereof.

A Synergistic Polyphenol Composition comprises synergistic amounts of two or more Synergistic Polyphenol Compounds. In one embodiment, a Synergistic Polyphenol Composition may contain: (a) an amount of a first Synergistic Polyphenol Compound which is less than the amount of said Compound when said Compound is administered as a single-agent, (b) an amount of a second Synergistic Polyphenol Compound which is less than the amount of said Compound when said Compound is administered as a single-agent, and (c) an amount of one or more additional anticancer agents or pharmaceutically acceptable salts thereof, which is less than the amount of said additional anticancer agents when said additional anticancer agent is administered as a single-agent. In another embodiment, a synergistic combination may contain an amount of a first Synergistic Polyphenol Compound, an amount of a second Synergistic Polyphenol Compound, and an amount of one or more additional anticancer agents or pharmaceutically acceptable salts thereof, which is similar to the amounts used when each of these agents are administered alone for the treatment of cancer.

The Synergistic Polyphenol Compositions of the present invention can be in any form that allows for the composition to be administered to a subject.

When administered to a subject, a Synergistic Polyphenol Compound can be administered as a component of a composition that comprises a physiologically acceptable carrier or vehicle. In one embodiment, the composition further comprises an additional anticancer agent. The present compositions, which comprise a Synergistic Polyphenol Compound, can be administered orally. The compositions can also be administered by any other convenient route, for example, by infusion or bolus injection, by absorption through epithelial or mucocutaneous linings (e.g., oral, rectal, and intestinal mucosa, etc.) and can be administered together with another biologically active agent. Administration can be systemic or local. Various delivery systems are known, e.g., encapsulation in liposomes, microparticles, microcapsules, capsules, etc., and can be administered.

Methods of administration include, but are not limited to, intradermal, intramuscular, intraperitoneal, intravenous, subcutaneous, intranasal, epidural, oral, sublingual, intracerebral, intravaginal, transdermal, rectal, by inhalation, or topical, particularly to the ears, nose, eyes, or skin. In some instances, administration will result in the release of the Synergistic Polyphenol Compounds into the bloodstream. The mode of administration is left to the discretion of the practitioner.

In one embodiment, the Synergistic Polyphenol Compounds or Synergistic Polyphenol Compositions are administered orally.

In another embodiment, the Synergistic Polyphenol Compounds or Synergistic Polyphenol Compositions are administered intravenously.

In other embodiments, it can be desirable to administer the Synergistic Polyphenol Compounds or Synergistic Polyphenol Compositions locally. This can be achieved, for example, and not by way of limitation, by local infusion during surgery, topical application, e.g., in conjunction with a wound dressing after surgery, by injection, by means of a catheter, by means of a suppository or enema, or by means of an implant, said implant being of a porous, non-porous, or gelatinous material, including membranes, such as sialastic membranes, or fibers.

In certain embodiments, it can be desirable to introduce the Synergistic Polyphenol Compounds or Synergistic Polyphenol Compositions into the central nervous system or gastrointestinal tract by any suitable route, including intraventricular, intrathecal, and epidural injection, and enema. Intraventricular injection can be facilitated by an intraventricular catheter, for example, attached to a reservoir, such as an Ommaya reservoir.

Pulmonary administration can also be employed, e.g., by use of an inhaler of nebulizer, and formulation with an aerosolizing agent, or via perfusion in a fluorocarbon or a synthetic pulmonary surfactant. In certain embodiments, the Synergistic Polyphenol Compounds or Synergistic Polyphenol Compositions can be formulated as a suppository, with traditional binders and excipients such as triglycerides.

In another embodiment the Synergistic Polyphenol Compounds or Synergistic Polyphenol Compositions can be delivered in a vesicle, in particular a liposome (see Langer, Science 249:1527-1533 (1990) and Treat or prevent et al., Liposomes in the Therapy of Infectious Disease and Cancer 317-327 and 353-365 (1989)).

In yet another embodiment the Synergistic Polyphenol Compounds or Synergistic Polyphenol Compositions can be delivered in a controlled-release system or sustained-release system (see, e.g., Goodson, in Medical Applications of Controlled Release, supra, vol. 2, pp. 115-138 (1984)). Other controlled or sustained-release systems discussed in the review by Langer, Science 249:1527-1533 (1990) can be used. In one embodiment a pump can be used (Langer, Science 249:1527-1533 (1990); Sefton, CRC Crit. Ref. Biomed. Eng. 14:201 (1987); Buchwald et al., Surgery 88:507 (1980); and Saudek et al., N. Engl. J. Med. 321:574 (1989)). In another embodiment polymeric materials can be used (see Medical Applications of Controlled Release (Langer and Wise eds., 1974); Controlled Drug Bioavailability, Drug Product Design and Performance (Smolen and Ball eds., 1984); Ranger and Peppas, J. Macromol. Sci. Rev. Macromol. Chem. 2:61 (1983); Levy et al., Science 228:190 (1935); During et al., Ann. Neural. 25:351 (1989); and Howard et al., J. Neurosurg. 71:105 (1989)).

In yet another embodiment a controlled- or sustained-release system can be placed in proximity of a target of the Synergistic Polyphenol Compounds or Synergistic Polyphenol Compositions, e.g., the spinal column, brain, heart, abdomen, thoracic cavity, skin, lung, or gastrointestinal tract, thus requiring only a fraction of the systemic dose.

The present compositions can optionally comprise a suitable amount of a physiologically acceptable excipient so as to provide the form for proper administration to the subject.

Such physiologically acceptable excipients can be liquids, such as water and oils, including those of petroleum, subject, vegetable, or synthetic origin, such as peanut oil, soybean oil, mineral oil, sesame oil and the like. The physiologically acceptable excipients can be saline, gum acacia; gelatin, starch paste, talc, keratin, colloidal silica, urea and the like. In addition, auxiliary, stabilizing, thickening, lubricating, and coloring agents can be used. In one embodiment the physiologically acceptable excipients are sterile when administered to a subject. Water is a particularly useful excipient when the composition is administered intravenously. Saline solutions and aqueous dextrose and glycerol solutions can also be employed as liquid excipients, particularly for injectable solutions. Suitable excipients also include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The present compositions, if desired, can also contain minor amounts of wetting or emulsifying agents, or pH buffering agents.

The present compositions can take the form of solutions, suspensions, emulsions, tablets, pills, pellets, capsules, capsules containing liquids, powders, sustained-release formulations, suppositories, aerosols, sprays, or any other form suitable for use. In one embodiment the composition is in the form of a capsule (see e.g. U.S. Pat. No. 5,698,155). Other examples of suitable physiologically acceptable excipients are described in Remington's Pharmaceutical Sciences 1447-1676 (Alfonso R. Gennaro eds., 19th ed. 1995), incorporated herein by reference.

In one embodiment the Synergistic Polyphenol Compounds or Synergistic Polyphenol Compositions are formulated in accordance with routine procedures as a composition adapted for oral administration to human beings. Compositions for oral delivery can be in the form of tablets, lozenges, aqueous or oily suspensions, granules, powders, emulsions, capsules, syrups, or elixirs for example. Orally administered compositions can contain one or more agents, for example, sweetening agents such as fructose, aspartame or saccharin; flavoring agents such as peppermint, oil of wintergreen, or cherry; coloring agents; and preserving agents, to provide a pharmaceutically palatable preparation. Moreover, where in tablet or pill form, the compositions can be coated to delay disintegration and absorption in the gastrointestinal tract thereby providing a sustained action over an extended period of time. Selectively permeable membranes surrounding an osmotically active platform driving a Synergistic Polyphenol Composition are also suitable for orally administered compositions. In these latter platforms, fluid from the environment surrounding the capsule is imbibed by the driving compound, which swells to displace the agent or agent composition through an aperture. These delivery platforms can provide an essentially zero order delivery profile as opposed to the spiked profiles of immediate release formulations. A time-delay material such as glycerol monostearate or glycerol stearate can also be used. Oral compositions can include standard excipients such as mannitol, lactose, starch, magnesium stearate, sodium saccharin, cellulose, and magnesium carbonate. In one embodiment the excipients are of pharmaceutical grade.

In another embodiment the compositions can be formulated for intravenous administration. Typically, compositions for intravenous administration comprise sterile isotonic aqueous buffer. Where necessary, the compositions can also include a solubilizing agent. Compositions for intravenous administration can optionally include a local anesthetic such as lignocaine to lessen pain at the site of the injection. Generally, the ingredients are supplied either separately or mixed together in unit dosage form, for example, as a dry lyophilized-powder or water free concentrate in a hermetically sealed container such as an ampule or sachette indicating the quantity of active agent. Where the compositions are to be administered by infusion, they can be dispensed, for example, with an infusion bottle containing sterile pharmaceutical grade water or saline. Where the compositions are administered by injection, an ampule of sterile water for injection or saline can be provided so that the ingredients can be mixed prior to administration.

The compositions can be administered by controlled-release or sustained-release means or by delivery devices that are well known to one skilled in the art. Examples include, but arc not limited to, those described in U.S. Pat. Nos. 3,845,770; 3,916,899; 3,536,809; 3,598,123; 4,008,719; 5,674,533; 5,059,595; 5,591,767; 5,120,548; 5,073,543; 5,639,476; 5,354;556; and 5,733,556, each of which is incorporated herein by reference. Such dosage forms can be used to provide controlled- or sustained-release of one or more active components using, for example, hydropropylmethyl cellulose, other polymer matrices, gels, permeable membranes, osmotic systems, multilayer coatings, microparticles, liposomes, microspheres, or a combination thereof to provide the desired release profile in varying proportions. Suitable controlled- or sustained-release formulations known to one skilled in the art, including those described herein, can be readily selected for use with the active components of the invention. The invention thus encompasses single unit dosage forms suitable for oral administration such as, but not limited to, tablets, capsules, gelcaps, and caplets that are adapted for controlled- or sustained-release.

In one embodiment a controlled- or sustained-release composition comprises a minimal amount of one or more Synergistic Polyphenol Compounds so as to treat or prevent cancer in a minimal amount of time. Advantages of controlled- or sustained-release compositions include extended activity of the drug, reduced dosage frequency, and increased subject compliance. In addition, controlled- or sustained-release compositions can favorably affect the time of onset of action or other characteristics, such as blood levels of the Synergistic Polyphenol Compounds, and can thus reduce the occurrence of adverse side effects.

Controlled- or sustained-release compositions can initially release an amount of a Synergistic Polyphenol Compound that promptly produces the desired therapeutic or prophylactic effect, and gradually and continually release other amounts of the Synergistic Polyphenol Compounds to maintain this level of therapeutic or prophylactic effect over an extended period of time. To maintain a constant level of a Synergistic Polyphenol Compound in the body, the Synergistic Polyphenol Compound can be released from the dosage form at a rate that will replace the amount of Synergistic Polyphenol Compound being metabolized and excreted from the body. Controlled- or sustained-release of a Synergistic Polyphenol Composition or a Synergistic Polyphenol Compound component of a Synergistic Polyphenol Composition can be stimulated by various conditions, including but not limited to, changes in pH, changes in temperature, concentration or availability of enzymes, concentration or availability of water, or other physiological conditions or compounds.

In one embodiment, the Synergistic Polyphenol Compositions comprise an amount of each the Synergistic Polyphenol Compounds which together are effective to treat cancer. In another embodiment, the Synergistic Polyphenol Compositions comprise an amount of the Synergistic Polyphenol Compounds which are effective to treat cancer when each of the compounds are administered separately as monotherapy. Typically, the compositions of the invention comprise at least about 0.01% of the combined Synergistic Polyphenol Compounds by weight of the composition. When intended for oral administration, this amount can be varied to be between 0.1% and 80% by weight of the composition. In one embodiment, an oral composition can comprise from between 4% and 50% of combined amount of the Synergistic Polyphenol Compounds by weight of the composition. In another embodiment, compositions of the present invention are prepared so that a parenteral dosage unit contains from between 0.01% and 2% by weight of the combined amount of the Synergistic Polyphenol Compounds of the invention.

A Synergistic Polyphenol Compound can administered to a subject at dosages from about 1 mg/m² to about 1000 mg/m², from about 100 mg/m² to about 700 mg/m², or from about 200 mg/m² to about 500 mg/m². The dosage administered is dependent upon various parameters, including, but not limited to, the cancer being treated, the subject's general health, and the administering physician's discretion. In specific embodiments, the total combined dosage of all Synergistic Polyphenol Compounds administered to a subject is about 50 mg/m², about 75 mg/m², about 100 mg/m², about 125 mg/m², about 150 mg/m², about 175 mg/m², about 200 mg/m², about 225 mg/m², about 250 mg/m², about 275 mg/m², about 300 mg/m², about 325 mg/m², about 350 mg/m², about 375 mg/m², about 400 mg/m², about 425 mg/m², about 450 mg/m², about 475 mg/m², about 500 mg/m², about 525 mg/m², about 550 mg/m², about 575 mg/m², about 600 mg/m², about 625 mg/m², about 650 mg/m², about 675 mg/m², about 700 mg/m², about 725 mg/m², about 750 mg/m², about 775 mg/m², about 800 mg/m², about 825 mg/m², about 850 mg/m², about 875 mg/m², about 900 mg/m², about 925 mg/m², about 950 mg/m², about 975 mg/m², or about 1000 mg/m².

The amount of the Synergistic Polyphenol Compounds that is effective in the treatment or prevention of cancer can be determined by standard clinical techniques. In addition, in vitro or in vivo assays can optionally be employed to help identify optimal dosage ranges. The precise dose to be employed will also depend on the identity of the Synergistic Polyphenol Compounds being administered, route of administration, and the seriousness of the condition being treated and should be decided according to the judgment of the practitioner and each subject's circumstances in view of, e.g., published clinical studies. Suitable effective amounts for each Synergistic Polyphenol Compound being administered, however, range from about 10 micrograms to about 5 grams. In certain embodiments, the effective amount is about 50 mg, about 100 mg, about 200 mg, about 300 mg, about 400 mg, about 500 mg, about 600 mg, about 700 mg, about 800 mg, about 900 mg, about 1 g, about 1.2 g, about 1.4 g, about 1.6 g, about 1.8 g, about 2.0 g, about 2.2 g, about 2.4 g, about 2.6 g, about 2.8 g, and about 3.0 g. Dosages may be administered over various time periods including, but not limited to, about every 2 hours, about every 6 hours, about every 8 hours, about every 12 hours, about every 24 hours, about every 36 hours, about every 48 hours, about every 72 hours, about every week, about every two weeks, about every three weeks, about every month, and about every two months.

In one embodiment, the Synergistic Polyphenol Compounds are administered concurrently to a subject in separate compositions. The Synergistic Polyphenol Compounds may be administered to a subject by the same or different routes of administration.

When the Synergistic Polyphenol Compounds are administered to a subject concurrently, the term “concurrently” is not limited to the administration of the Synergistic Polyphenol Compounds at exactly the same time, but rather it is meant that they are administered to a subject in a sequence and within a time interval such that they can act synergistically to provide an increased benefit than if they were administered otherwise. For example, the Synergistic Polyphenol Compounds may be administered at the same time or sequentially in any order at different points in time; however, if not administered at the same time, they should be administered sufficiently close in time so as to provide the desired therapeutic effect, preferably in a synergistic fashion. The Synergistic Polyphenol Compounds can be administered separately, in any appropriate form and by any suitable route. When the Synergistic Polyphenol Compounds are not administered in the same composition, it is understood that they can be administered in any order to a subject in need thereof. For example, a first Synergistic Polyphenol Compound can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second Synergistic Polyphenol Compound, to a subject in need thereof. In various embodiments the Synergistic Polyphenol Compounds are administered concurrently, 1 minute apart, 10 minutes apart, 30 minutes apart, less than 1 hour apart, 1 hour apart, 1 hour to 2 hours apart, 2 hours to 3 hours apart, 3 hours to 4 hours apart, 4 hours to 5 hours apart, 5 hours to 6 hours apart, 6 hours to 7 hours apart, 7 hours to 8 hours apart, 8 hours to 9 hours apart, 9 hours to 10 hours apart, 10 hours to 11 hours apart, 11 hours to 12 hours apart, no more than 24 hours apart or no more than 48 hours apart. In one embodiment, the Synergistic Polyphenol Compounds are administered within the same office visit. In another embodiment, the Synergistic Polyphenol Compounds are administered at 1 minute to 24 hours apart.

Suitable effective dosage amounts for the Synergistic Polyphenol Compositions are based upon the total amount of the Synergistic Polyphenol Compounds present in the composition. For the Synergistic Polyphenol Compositions of the present, the total amount of Synergistic Polyphenol Compounds should be within a range of from about 0.01 to about 100 w/w. The effective dosage amounts described herein refer to the total amounts of all Synergistic Polyphenol Compounds administered. If one or more Synergistic Polyphenol Composition is administered, the effective dosage amounts correspond to the combined amount of all Synergistic Polyphenol Compounds in each of the Synergistic Polyphenol Compositions administered.

The Synergistic Polyphenol Compounds or Synergistic Polyphenol Compositions can be assayed in vitro or in vivo for the desired therapeutic or prophylactic activity prior to use in humans. Subject model systems can be used to demonstrate safety and efficacy.

The present methods for treating or preventing cancer in a subject can further comprise administering another therapeutic agent to the subject being administered a Synergistic Polyphenol Compound or Synergistic Polyphenol Composition. In one embodiment the other therapeutic agent is administered in an effective amount.

Effective amounts of the other therapeutic agents are well known to one skilled in the art. However, it is well within the skilled artisan's purview to determine the other therapeutic agent's optimal effective amount range.

In one embodiment, the other therapeutic agent is an antiemetic agent.

In yet another embodiment the other therapeutic agent is a hematopoietic colony stimulating factor.

In a further embodiment, the other therapeutic agent is an agent useful for reducing any potential side effect of a Synergistic Polyphenol Composition, a Synergistic Polyphenol Compound, or another anticancer agent.

In one embodiment, the Synergistic Polyphenol Compounds or Synergistic Polyphenol Compositions can be administered prior to, at the same time as, or after an antiemetic agent, or on the same day, or within 1 hour, 2 hours, 12 hours, 24 hours, 48 hours or 72 hours of each other.

In another embodiment, the Synergistic Polyphenol Compounds or Synergistic Polyphenol Compositions can be administered prior to, at the same time as, or after a hematopoietic colony-stimulating factor, or on the same day, or within 1 hour, 2 hours, 12 hours, 24 hours, 48 hours, 72 hours, 1 week, 2 weeks, 3 weeks or 4 weeks of each other.

The Synergistic Polyphenol Compounds or Synergistic Polyphenol Compositions of the present invention can also be cyclically administered. Cycling therapy involves the administration of one Synergistic Polyphenol Compound or Synergistic Polyphenol Composition for a period of time, followed by the administration of a second Synergistic Polyphenol Compound or Synergistic Polyphenol Composition for a period of time and repeating this sequential administration, i.e., the cycle, in order to reduce the development of resistance to one or both of the Synergistic Polyphenol Compounds or Synergistic Polyphenol Compositions, to avoid or reduce the side effects of one or both of the Synergistic Polyphenol Compounds or Synergistic Polyphenol Compositions, and/or to improve the efficacy of the treatment.

In one embodiment, the cycling therapy includes one or more other anticancer agents in addition to the Synergistic Polyphenol Compounds or Synergistic Polyphenol Compositions.

In one embodiment, the Synergistic Polyphenol Compounds or Synergistic Polyphenol Compositions are administered concurrently to a subject in separate compositions. The Synergistic Polyphenol Compounds or Synergistic Polyphenol Compositions may be administered to a subject by the same or different routes of administration.

### 5.6 Kits

The invention encompasses kits that can simplify the administration of a Synergistic Polyphenol Compound or a Synergistic Polyphenol Composition to a subject.

A typical kit of the invention comprises a unit dosage form of a Synergistic Polyphenol Compound or a Synergistic Polyphenol Composition. In one embodiment the unit dosage form is a container, which can be sterile, containing an effective amount of a Synergistic Polyphenol Compound or a Synergistic Polyphenol Composition, and a physiologically acceptable carrier or vehicle. The kit can further comprise a label or printed instructions instructing the use of the Synergistic Polyphenol Compound or a Synergistic Polyphenol Composition to treat or prevent cancer in a subject. The kit can also further comprise a unit dosage form of another therapeutic agent, for example, a container containing an effective amount of the other therapeutic agent. In one embodiment the kit comprises a container containing an effective amount of a Synergistic Polyphenol Compound or a Synergistic Polyphenol Composition and an effective amount of another therapeutic agent. Examples of other therapeutic agents include, but are not limited to, those listed above.

Kits of the invention can further comprise a device that is useful for administering the unit dosage forms. Examples of such a device include, but are not limited to, a syringe, a drip bag, a patch, an inhaler, and an enema bag.

The following examples are set forth to assist in understanding the invention and should not, of course, be construed as specifically limiting the invention described and claimed herein. Such variations of the invention, including the substitution of all equivalents now known or later developed, which would be within the purview of one skilled in the art, and changes in formulation or minor changes in experimental design, are to be considered to fall within the scope of the invention incorporated herein.

### 6. EXAMPLES

Chemicals. (−)-epigallocatechin gallate and Poly E were provided by the National Cancer Institute (Bethesda, Md.). Poly E contains about 60% (−)-epigallocatechin gallate, 7% (−)-epicatechin, 12% EGC, 1% (−)-epicatechin gallate, and 2% (−)-gallocatechin gallate, respectively.

Cell lines and cell culture. The Caco2, HCT116, HT29, SW480, and SW837 human colorectal cancer cell lines and the FHC normal human fetal colon cell line were obtained from American Type Culture Collection (Manassas, Va.). All cell lines were maintained in DF10 medium containing DMEM (Invitrogen, San Diego, Calif.) supplemented with 10% fetal bovine serum (Invitrogen). The FHC cell line was originally established from the colon of a human fetus at 13 weeks of gestation and has an epithelial morphology. Cells were cultured in an incubator with humidified air at 37° C. with 5% CO₂. As an untreated solvent control, cells were treated with DMSO (Sigma Chemical Co., St. Louis, Mo.) at a final concentration of <0.1%.

### 6.1 Example 1

### Extraction of Polyphenols From Green Leaves

Crude polyphenols can be extracted from Chinese green tea leaves (500 g) using hot water, as described in European Patent No. EP 1402869 to Schneider, then dissolved in ethanol (5 mL). The ethanolic solution is then loaded onto a C16/100 chromatographic column (1.6 cm×90 cm, Sephadex LH-20, equilibrated using ethanol) and chromatographed using a flow rate of about 1.2 mL/min. Collected fractions can be first analyzed using thin-layer chromatography on silica gel plates (chloroform/methanol/water (65:35:10, v/v/v as eluent) and subsequently developing the eluted plates using a spray reagent that is prepared by dissolving 1 g vanillin in 50 mL concentrated HCl. Fractions that are positive to the spray reagent can be concentrated in vacuo, and the resulting residue dissolved in methanol. The resulting methanolic solution can be analyzed using UV/visible spectroscopy by measuring their absorbances at 280 nm and 500 nm for detecting the presence of polyphenols. Fractions containing crude polyphenols can be purified using the methods described in Hoefler et al., J. Chromatogr. 129:460-3 (1976).

### 6.2 Example 2

### Cell Viability Assay

Cell viability assays were performed as described in Suzui et al., Cancer Res. 2002; 62:3997-4006, using the 3-(4,5-dimethylthiazol-2-yl)-2,5-diphenyltetrazolium bromide (MTT) cell proliferation kit I (Roche Diagnostics Co., Indianapolis, Ind.), according to the instructions of the manufacturer. Cells to be tested against were plated onto 96-well plates (3×10³ cells/well) and twenty-four hours later, the cells were treated with an illustrative Synergistic Polyphenol Composition, or one or more illustrative Synergistic Polyphenol Compounds. Neither the medium nor the test compounds were changed during this time period. The assay was done in triplicate.

### 6.3 Example 3

### Determination of the Synergistic Anticancer Properties of Selected Synergistic Polyphenol Compounds Against a Human Colorectal Cancer Cell Line

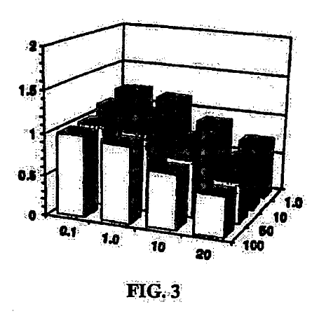

Three thousand HT29 cells were plated onto 96-well plates. Twenty-four hours later, (−)-epigallocatechin gallate and (−)-epicatechin were added alone or in combination at the concentrations indicated above in Example 2 to each well, and the cells were incubated for 48 hours in DF10 medium. cell viability assays were then done using the MTT system as described in Example 2. To determine whether the combined effects of (−)-epigallocatechin gallate plus (−)-epicatechin were synergistic, the combination index isobologram was used to analyze the data obtained as described, for example, in Soriano et al., Cancer Res. 1999; 59:6178-84 and Shimizu et al., Clin. Cancer Res. 2004;10:6710-2. An isobologram corresponding to the collected data is shown in FIG. 3, while Table 3 summarizes the results of the isobologram analysis.

TABLE 3
Summary of the Isobologram Analysis of the Effect of Combinations of
(−)-epigallocatechin gallate and (−)-epicatechin on the Viability
of a Human Colorectal Cancer Cell Line

| | |
|---|---|
| (−)-epicatechin | (−)-epigallocatechin gallate |
| concentration | concentration (μg/mL) |

| (μg/mL) | 0.1 | 1 | 10 | 20 |
|---|---|---|---|---|
| 1 | − | − | + | ++ |
| 10 | − | ± | + | ++ |
| 50 | ± | ± | ++ | +++ |
| 100 | ± | ± | ++ | +++ |

− indicates no synergy
± indicates a moderate additive effect
+ indicates slight synergism
++ indicates moderate synergism
+++ indicates synergism

These results indicate that a combination of the Synergistic Polyphenol Compounds (−)-epigallocatechin gallate and (−)-epicatechin causes synergistic inhibitions of growth in a human cancer cell line. Accordingly, the Synergistic Polyphenol Compounds or Synergistic Polyphenol Compositions are useful for treating colorectal cancer.

### 6.4 Example 4

### Determination of the Anti-Proliferative Effects of Illustrative Synergistic Polyphenol Compounds in a Human Colorectal Cancer Cell Line

One hundred thousand HT29 cells were seeded in multiple 35-mm diameter dishes in DF10 medium, and 24 hours later they were treated with (−)-epigallocatechin gallate or Poly E for 96 hours at a concentration of 1 or 20 μg/mL. The medium and test compounds were not changed during this time period. As a control, the cells were treated with 0.1% DMSO. Every 24 hours up to 96 hours, replicate plates of cells were washed with phosphate-buffered saline and the attached cells were collected using trypsinization. The numbers of cells in replica plates were then determined using a Coulter Counter (Beckman Coulter Co., Fullerton, Calif.) as described in Shimizu et al., Clin. Cancer Res. 2004: 10:1130-40. Table 4 shows IC₅₀ values for both (−)-epigallocatechin gallate and Poly E against various human colorectal cancer cell lines.

TABLE 4
IC50 values for (−)-epigallocatechin gallate and Poly E against
human colorectal cancer cell lines

| | IC₅₀ | | | |
|---|---|---|---|---|
| | (−)- | (−)- | | |
| | epigallocatechin | epigallocatechin | | |
| Cell | gallate | gallate | Poly E | Poly E |
| Line | (μg/mL) | (μmol/L) | (μg/mL) | (μmol/L) |
| Caco2 | 19.2 | 42.2 | 22.3 | 29.4 |
| HCT116 | 21.7 | 47.7 | 20.6 | 27.2 |
| HT29 | 22.8 | 50.2 | 20.1 | 26.5 |
| SW480 | 36.4 | 80.1 | 37.0 | 48.4 |
| SW837 | 19.6 | 43.1 | 19.6 | 25.9 |
| FHC | 48.8 | 107.4 | 49.4 | 65.2 |

These results suggest that ECGC and Poly E preferentially inhibit the growth of human colorectal cancer cells and accordingly, the Synergistic Polyphenol Compounds or Synergistic Polyphenol Compositions are useful for treating colorectal cancer.

### 6.5 Example 5

### Induction of Apoptosis in a Human Colorectal Cancer Cell Line

To quantify the induction of apoptosis in human colorectal cancer cell lines, a DNA fragmentation assay can be performed using the Cell Death Detection ELISA^{PLUS} kit (Roche Diagnostics), as described in Shimizu et al., Clin. Cancer Res. 2004; 10:6710-2, and Shimizu et al., Clin. Cancer Res. 2004: 10:1130-40. In this assay, human colorectal cancer cell cells can be seeded into 6-cm diameter dishes (5×10⁵ cells/dish) in DF10 medium, and 24 hours later the cells are treated with a test compound for an appropriate time as set forth in the cited references.

### 6.6 Example 6

### Protein Extraction, Immunoprecipitation, and Western Blot Analysis

Total cellular protein was extracted and equivalent amounts of protein can be determined by western blot analysis using the methods described in Suzui, et al., Cancer Res., 62:3997-4006 (2002). The primary antibodies for several proteins are described in Masuda et al., Clin Cancer Res., 7:4220-4229 (2001); Masuda et al., Clin Cancer Res., 9:3486-3491 (2003); and Shimizu et al., Clin Cancer Res., 11:2735-2746 (2005). The primary antibodies for HER3 (2F12) and COX-2 (PG27) were purchased from Upstate Cell Signaling Solutions (Charlottesville, Va.) and Oxford Biochemical Research (Oxford, Mich.), respectively. To examine the levels of expression of the phosphorylated (i.e. activated) form of the HER3 protein (p-HER3), cellular lysates (1 mg/mL) were incubated with 4 μg anti-HER3 antibody overnight at 4° C. with slow agitation, and immune complexes were then precipitated by adding protein A/G agarose (sc-2003, Santa Cruz Biotechnology, Santa Cruz, Calif.). After incubating for 2 hours at 4° C., the beads were washed with lysis buffer, and bound proteins were eluted into the sample buffer and subjected to western blot analysis, as described above. The p-HER3 protein was then detected by using an anti-phospho-tyrosine antibody (PY-20-HRP, BD Transduction Laboratories, San Diego, Calif.). An antibody to actin was used as a loading control. Each membrane was developed using an ECL-enhanced chemiluminescence system (Amersham Biosciences, Piscataway, N.J.).

### 6.7 Example 7

### RNA Extraction and Semiquantitative RT-PCR Analysis

RNA extraction and semiquantitative RT-PCR analysis were performed using the methods described in Suzui, et al., Cancer Res., 62:3997-4006 (2002). Total RNA was isolated from SW837 human colon cancer cells using Trizol reagent (Invitrogen) according to the manufacturer's instructions. cDNA was amplified from 1 μg of total RNA using SuperScript one-step RT-PCR with the platinum Taq system (Invitrogen). PCR was conducted for 25 cycles in a thermal controller (Programmable Thermal Controller; MJ Research Inc., Watertown, Mass.). The COX-2 specific primer sets F1COX-2 (5′-GTT CCC ACC CAT GTC AAA AC-3′) and R2COX-2 (5′-GCC CTC GCT TAT GAT CTG TC-3′) were designed using the sequence described in Hla et al., Proc. Natl. Acad. Sci. USA, 89:7384-7388 (2004). Actin specific primer sets and the amplification cycle conditions were as described in Suzui, et al., Cancer Res., 62:3997-4006 (2002). The intensities of mRNA bands were quantified using NIH Image software version 1.62.

### 6.8 Example 8

### DNA Fragmentation Assays

To quantify the induction of apoptosis, a DNA fragmentation assay was performed using the Cell Death Detection ELISA^{PLUS} kit (Roche Diagnostics Co., Indianapolis, Ind.), according to the manufacturer's instructions and as described in Shimizu et al., Clin. Cancer Res., 10:1130-1140 (2004). SW837 human colon cancer cells (5.0×10⁵ cells/6-cm diameter dish) were treated with EGCG (1.0 or 20 μg/mL) for 48 or 96 hours, and samples were then prepared. Control cells were treated only with DMSO. The extent of DNA fragmentation at time 0 hours was set to 1 and increases expressed as fold activation.

### 6.9 Example 9

### COX-2, AP-1, and NF-κB Reporter Assays

Reporter assays were performed performed using the methods described in Suzui, et al., Cancer Res., 62:3997-4006 (2002). The COX-2-luciferase reporter plasmid phPES2(−327/+59) was provided by the National Cardiovascular Research Institute (Osaka, Japan). The pAP-1 luciferase reporter plasmid was provided by the National Cancer Institute (Bethesda, Md.). The NF-□B promoter luciferase reporter plasmid □NF-□B-Luc is described in Masuda et al., Clin. Cancer Res. 7:4220-4229 (2001). One μg of DNA of the indicated luciferase reporter plasmid was transfected into SW837 human colon cancer cells (3.0×10⁵ cells/35-mm diameter dish) using Lipofectin reagent (Invitrogen) in opti-MEM I medium (Invitrogen). After 24 hours the cells were treated with EGCG (0, 10, or 20 μg/mL) for 24 hours, and extracts were then prepared. Luciferase activities were determined using a luciferase assay system (Program Co., Madison, Wis.). In all of these reporter assays the cells were also cotransfected with a CMV-□-galactosidase (□-gal) reporter. Luciferase activities were normalized with respect to the □-gal activities, to correct for differences in transfection efficiency, using the methods described in Suzui, et al., Cancer Res., 62:3997-4006 (2002).

### 6.10 Example 10

### PGE₂ Production Assays

SW837 human colon cancer cells were plated into 35-mm dishes and grown to 60% confluence. The cells were then treated with EGCG (0, 10, 20, or 40 μg/mL) in serum minus medium containing 20 μM arachidonic acid (Sigma) for 18 hours. The cell free medium was then collected and the amounts of PGE₂ released by cells into the medium were measured using an EIA kit (Cayman Chemicals, Ann Arbor, Mich.), according to the manufacturer's instructions. Production of PGE₂ was normalized to the protein concentration of the cells.

### 6.11 Example 11

### Cell Proliferation Assays

Thirty thousand SW837 human colon cancer cells were seeded into multiple 35-mm diameter dishes in DFI0 medium, and 24 hours later the cells were treated with EGCG (0, 1.0, or 20 μg/mL) for 96 hours, during which time, the medium and drugs were not changed. As a control, the cells were treated with 0.1% DMSO. Every 24 hours, and for a total time of 96 hours, replicate plates of cells were washed with PBS and the attached cells were collected by trypsinization. The numbers of cells in replica plates were then counted using a Coulter Counter (Beckman Coulter Co., Fullerton, Calif.) as described in Shimizu et al., Clin. Cancer Res., 10:1130-1140 (2004).

### 6.12 Example 12

### Statistical Analysis

Statistical analyses of DNA fragmentation assays, reporter assays, and PGE₂ production assays were analyzed by Student's t test. The results were considered statistically significant if the P value was less than 0.05.

### 6.13 Example 13

### The COX-2 and HER3 Proteins are Overexpressed in SW837 and Caco2 Colon Cancer Cells

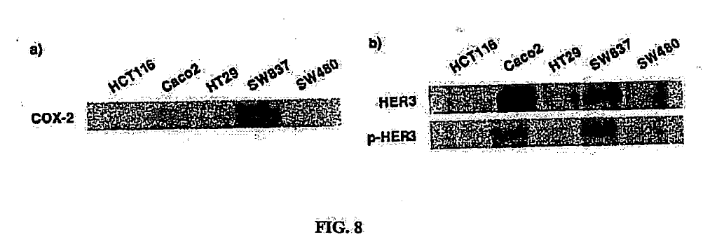

Using western blot analysis as described above in Example 6, it was determined that the level of the COX-2 protein was markedly increased in SW837 human colon cancer cells and was expressed at a moderate level in Caco2 cells, as shown in FIG. 8(a). The HER3 protein was also expressed at a high level in the SW837 and Caco2. The level of the p-HER3 protein was also increased in these two cell lines, indicating constitutive activation of this receptor, as shown in FIG. 8(b). It has been reported that the EGFR and HER2 proteins are also overexpressed and constitutively activated in SW837 human colon cancer cells, and that these cells display strong activation for EGFR, HER2, and HER3. Because activation of members of the EGF receptor family, especially the HER2/HER3 heterodimer, has been implicated in the induction of COX-2 expression in colon cancer cells, SW837 human colon cancer cells are thereby useful in studies involving HER2 and COX-2.

### 6.14 Example 14

### EGCG Inhibits Activation of EGFR, HER2, and HER3 Signaling Pathways, Inhibits Expression of COX-2, and Induces Apoptosis in SW837 human colon cancer cells

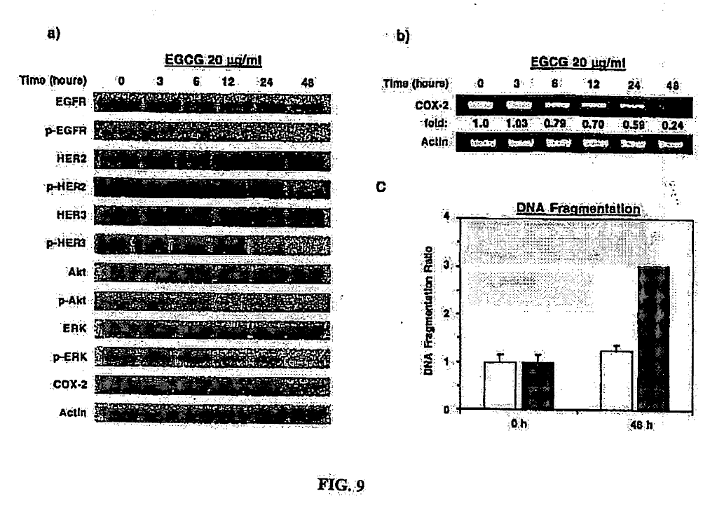

It is well-established that abnormalities in the EGFR family of RTKs play critical roles in the development of various types of cancer, and it is reported that the EGFR and HER2 receptors are targets of EGCG, an illustrative Synergistic Polyphenol Compound1, in a variety of cancer cell lines. It was examined in parallel whether EGCG, an illustrative Synergistic Polyphenol Compound, inhibits activation of EGFR, HER2, and HER3, and related downstream signaling pathways in SW837 colon cancer cells. Also examined was the effect of EGCG on cellular levels of both COX-2 protein (FIG. 9(a)) and mRNA (FIG. 9(b)) in SW837 human colon cancer cells. Time course studies indicate that when the cells were treated with 20 μg/mL EGCG, the IC₅₀ concentration determined by MTT assays as described in Shimizu et al., Clin. Cancer Res., 11:2735-2746 (2005), there was a marked decrease in the levels of the p-EGFR, p-HER2, and p-HER3 proteins within 6 hours after the addition of EGCG. The levels of the p-Akt and p-ERK proteins displayed marked decreases at 6 and 12 hours, respectively, after the addition of EGCG. During this period of time there were no significant changes in the total levels of the respective proteins, indicating that EGCG specifically inhibited activation of EGFR, HER2, and HER3; and also activation of the downstream signaling molecules Akt and ERK. Results indicate that the cellular level of the COX-2 protein displayed a marked decrease after 12 to 24 hours, and was almost undetectable at 48 hours, after treatment with EGCG, as shown in FIG. 9(a). Semiquantitative RT-PCR studies indicated that EGCG caused a progressive decrease in the cellular level of the COX-2 mRNA beginning at about 6 hours, with a marked decrease at 48 hours, as shown in FIG. 9(b). In addition, DNA fragmentation assays, performed as described in Example 8 above, indicate that treatment with EGCG for 48 hours caused induction of apoptosis in SW837 human colon cancer cells, as shown in FIG. 9(c). Accordingly, the Synergistic Polyphenol Compounds are useful for treating human colorectal cancer.

### 6.15 Example 15

### EGCG Inhibits Transcriptional Activity of the COX-2, AP-1, and NF-κB Promoters

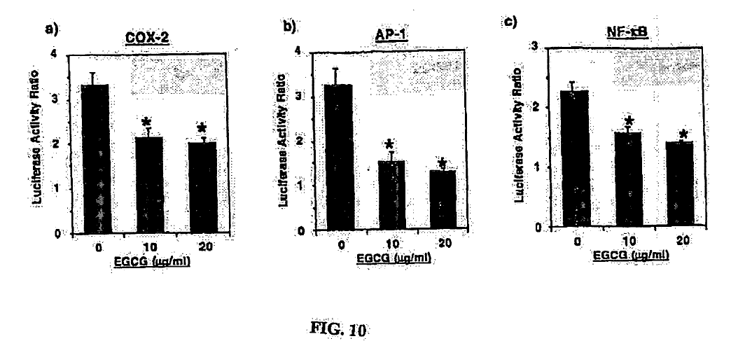

There is evidence that the transcription factor AP-1, which lies downstream of ERK, plays an important role in activating expression of COX-2 via the CRE element in the COX-2 promoter. Increased binding of AP-1 to CRE in the COX-2 promoter occurs in HER2 transformed human mammary epithelial cells. There is also evidence that the NF-□B signaling pathway, which is activated by the Akt pathway, plays an important role in regulating COX-2 expression via the NF-□B binding site in the COX-2 promoter. The effects of EGCG, an illustrative Synergistic Polyphenol Compound, on the transcriptional activity of the COX-2, AP-1, and NF-□B promoters, was determined using transient transfection luciferase reporter assays. As shown in FIG. 10, exposure of SW837 human colon cancer cells to EGCG (0, 10 and 20 μg/mL)caused inhibition of COX-2 (FIG. 10(a)), AP-1(B) (FIG. 10(b)), and NF-□B (FIG. 10(c)) luciferase reporter activities. EGCG induced inhibition was most apparent with respect to the AP-1 promoter activity. Taken together with the results of the study described in Example 14 above, which indicates that EGCG causes a decrease in cellular levels of both COX-2 protein and mRNA, these results provide evidence that the inhibitory effects of EGCG on expression of COX-2 are exerted, at least in part, at the level of gene transcription, presumably through inhibition of activation of the EGFR, HER2, and HER3 receptors, and downstream signaling molecules, such as ERK and Akt.

### 6.16 Example 16

### EGCG Causes a Decrease in the Production of PGE₂ by SW837 human colon cancer cells

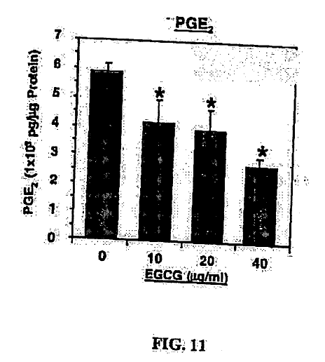

Treatment of SW837 human colon cancer cells with EGCG, an illustrative Synergistic Polyphenol Compound (0, 10, 20 or 40 μg/mL), resulted in a decrease in the levels of PGE₂, a major product of the COX-2 enzyme, as indicated in FIG. 11. Because PGE₂ can enhance the proliferation, migration, motility, and invasion of colon cancer cells, inhibition of the production of PGE₂ by EGCG may play a critical role in the ability of this compound to inhibit growth and induce apoptosis in colon cancer cells. Accordingly, the Synergistic Polyphenol Compounds are useful for the treatment of human colorectal cancer.

### 6.17 Example 17

### Longer Exposure to a Low Concentration of EGCG Inhibits Cell Growth, Induces Apoptosis, Inhibits Activation of the EGFR, HER2, and HER3 Receptors, and Inhibits Expression of COX-2 and Bcl-x_{L} Proteins in SW837 human colon cancer cells

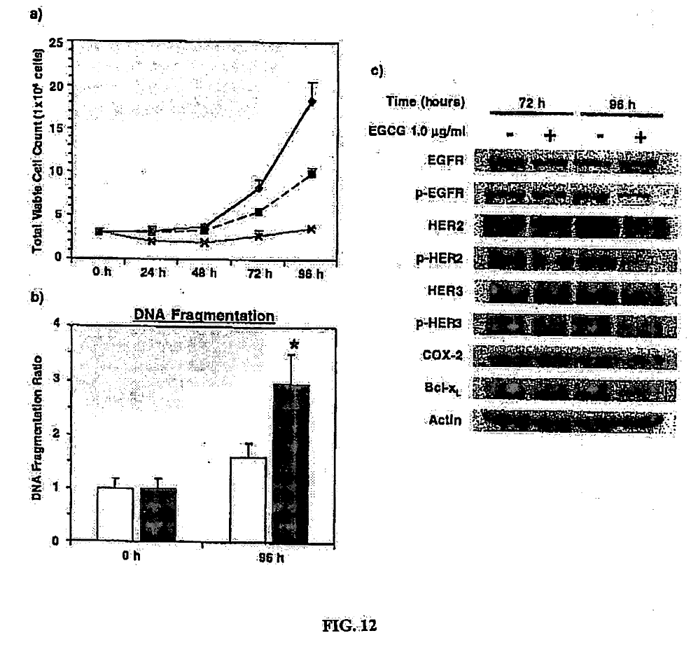

Using the method described above in Example 11, SW837 human colon cancer cells were treated with 1.0 μg/mL of EGCG, an illustrative Synergistic Polyphenol Compound, for 96 hours. This resulted in an inhibition of growth of about 55%, a significant increase in a decrease in the levels of the p-EGFR, p-HER2, p-HER3, COX-2, and Bcl-x_{L} proteins as indicated in FIG. 12(a), and a significant increase in apoptosis, as shown in FIG. 12(b). Accordingly, administration of a low dose of EGCG may inhibit activation of these RTKs, the expression of COX-2, and the growth of human colon cancer cells. As such, the Synergistic Polyphenol Compounds are useful for the treatment of human colorectal cancer.

### 6.18 Example 18

### Protein Extraction and Western Blot Analysis for IGF

Total cellular protein was extracted and equivalent amounts of protein were examined by western blot analysis, as described above in Example 6. Cell lysates were separated by SDS-PAGE using 7.5 to 15% polyacrylamide gels and transferred onto Immobilon-P transfer membranes (Millipore Co., Bedford, Mass.). The primary antibodies for IGF-1R□(#3022) and p-IGF-1R (#3021) were purchased from Cell Signaling Technology (Beverly, Mass.). The primary antibodies for IGF-1R□(sc-713) and IGFBP-3 (sc-6003) were purchased from Santa Cruz Biotechnology (Santa Cruz, Calif.). The primary antibodies for IGF-1 (clone Sm1.2) and actin (A2066) were purchased from Upstate Cell Signaling Solutions (Charlottesville, Va.) and Sigma Chemical Co. (St Louis, Mo.), respectively. An antibody to actin was used as a loading control. Anti-mouse IgG (Amersham Pharmacia Biosciences, Piscataway, N.J.), anti-rabbit IgG (Amersham Pharmacia Biosciences), or anti-goat IgG (Santa Cruz Biotechnology) antibodies were used as the secondary antibodies. Each membrane was developed using an ECL-enhanced chemiluminescence system (Amersham Biosciences).

### 6.19 Example 19

### RNA Extraction and Semiquantitative RT-PCR Analysis

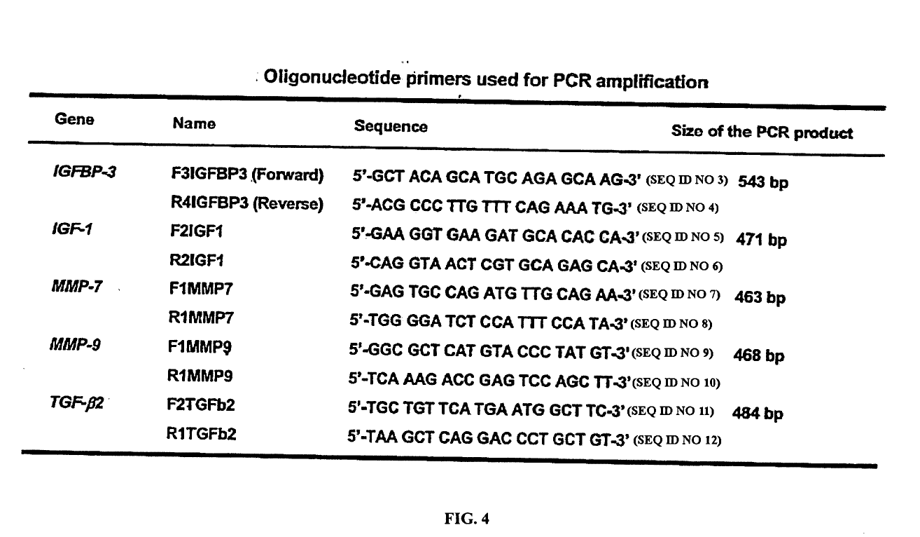

RNA extraction and semiquantitative RT-PCR analysis were performed as described above in Example 7. Total RNA was isolated from SW837 human colon cancer cells using the Trizol reagent (Invitrogen) according to the manufacturer's instructions. cDNA was amplified from 1 μg of total RNA using Superscript one-step RT-PCR with the platinum Taq system (Invitrogen). The primers used for amplification of IGFBP-3, IGF-1, MMP-7, MMP-9, and TGF-□2 specific genes are shown in FIG. 4. Actin specific primer sets are described in Suzui, et al., Cancer Res., 62:3997-4006 (2002). By using a thermal controller (Programmable Thermal Controller; MJ Research Inc., Watertown, Mass.), 25-, 35-, 32-, 40-, and 30-cycle rounds of PCR were chosen for optimum analysis of expression of IGFBP-3, IGF-1, MMP-7, MMP-9, and TGF-□2 mRNAs, respectively, since a semiquantitative assessment indicated that the reactions had not reached a plateau and were still in the log phase. Each amplification cycle consisted of 0.5 minutes at 94° C. for denaturation, 0.5 minutes at 55° C. for primer annealing, and 1 minute at 72° C. for extension. After PCR amplification, the fragments were analyzed by agarose gel electrophoresis and stained with ethidium bromide. The intensities of bands were quantified with NIH Image software version 1.62.

### 6.20 Example 20

### Expression of IGF-1R, p-IGF-1R, IGF-1, and IGFBP-3 Proteins in Colon Cancer Cell Lines

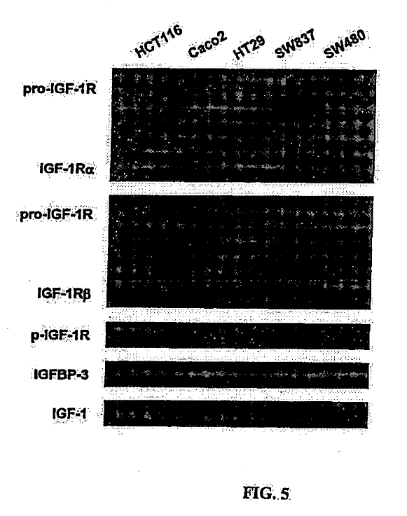

Western blot analysis was performed to determine the cellular levels of the IGF-1R, p-IGF-1R, IGF-1, and IGFBP-3 proteins in five human colon cancer cell lines (HCT116, Caco2, HT29, SW837 and SW480). Results indicate that the levels of the IGF-1R□and IGF-1R□proteins are increased in the Caco2, HT29, SW837, and SW480 cell lines, especially in SW837 and SW480 cells. The slower migrating pro-IGF-1R protein was also increased in the SW837 and SW480 cell lines. The level of the p-IGF-1R protein was also increased in the SW837 and SW480 cell lines, indicating constitutive activation of this receptor. All cell lines expressed the IGFBP-3 and IGF-I proteins at approximately similar levels, as illustrated in FIG. 5.

### 6.21 Example 21

### Effects of EGCG on Expression of IGF-1R and Related Proteins in SW837 Human Colon Cancer Cells

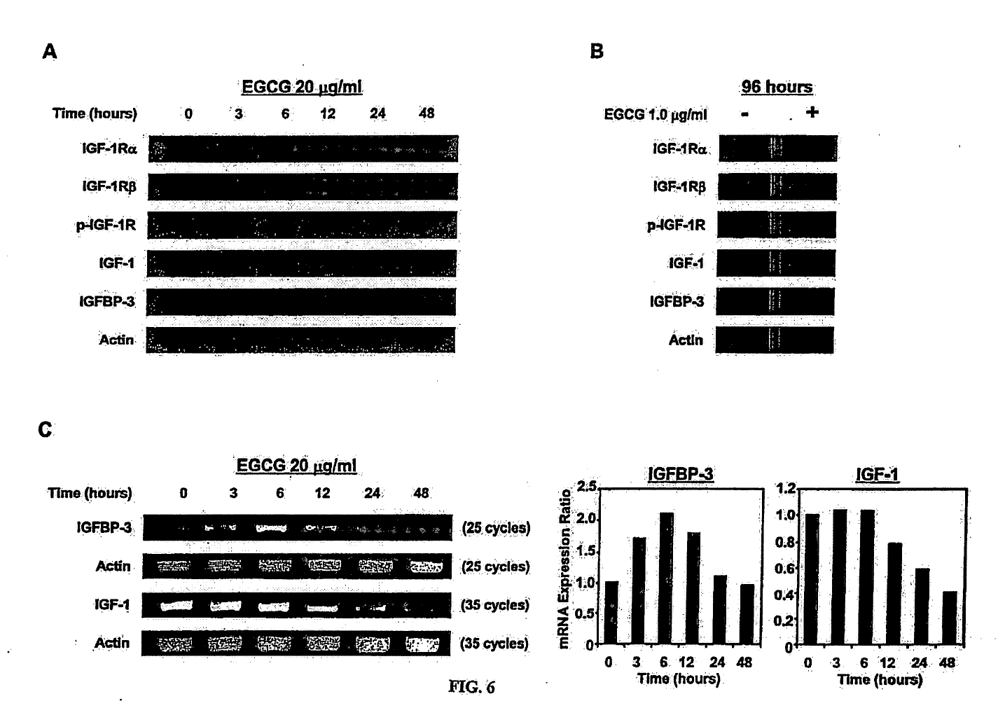

Abnormalities in the IGF-1/IGF-1R system play a role an important role in the development of various types of cancer. Since EGCG can inhibit activation of several RTKs including EGFR, HER2, and PDGF, the possible effects of this compound on activation of IGF-1R and on related proteins in SW837 colon cancer cells was determined. Time course studies indicated that when the cells were treated with 20 μg/mL EGCG, the IC₅₀ concentration determined using MTT assay, there was a marked decrease in the levels of the p-IGF-1R protein within 6 hours after the addition of EGCG, as shown in FIG. 6A. The levels of the IGF-1 protein displayed a moderate decrease at 12 hours and a marked decrease at 24 hours after the addition of EGCG. Thus, these decreases were delayed compared with the decrease in the levels of the p-IGF-1R protein. There was a marked increase in the level of the IGFBP-3 protein at 6 hours after the addition of this compound, and this increase persisted for at least 48 hours. During this period of time there were no significant changes in the levels of the IGF-1R□and IGF-1R□proteins, indicating that EGCG specifically inhibited activation of IGF-1R, but did not inhibit the total amount of these proteins as indicated in FIG. 6A.

It was then determined whether exposure to a low concentration (1.0 μg/mL) of EGCG, which is in the range of the peak plasma concentration of EGCG obtained in a clinical study, for a longer time period can also alter the levels of p-IGF-1R, IGF-1, and IGFBP-3 proteins in SW837 human colon cancer cells, as indicated in FIG. 6B. Results indicate that when the cells were treated with 1.0 μg/mL of EGCG for 96 hours, there was a decrease in the levels of the p-EGFR and IGF-1 proteins and an increase in the IGFBP-3 protein (FIG. 6B), thus reproducing the effects obtained EGCG at 20 μg/mL, as shown in FIG. 6A.

### 6.22 Example 22

### Effects of EGCG on Expression of IGFBP-3 and IGF-1 mRNAs in SW837 Colon Cancer Cells

To determine whether the specific changes in cellular levels of the IGF-1 and IGFBP-3 proteins induced by EGCG shown in FIG. 9A might be secondary to effects on expression of the corresponding genes, the effects of EGCG on the levels of IGFBP-3 and IGF-1 mRNAs were examined using semiquantitative RT-PCR analysis. Time course studies indicated that treatment with 20 μg/mL EGCG resulted an increase in the levels of IGFBP-3 mRNA at 3 to 12 hours after the addition of this compound, and the maximum level peaked at 6 hours. Results indicate that there is a decrease in the level of IGF-1 mRNA at 12 hours and an additional decline at 24 and 48 hours, as shown in FIG. 6C.

### 6.23 Example 23

### Effects of EGCG on Expression of MMPs-7 and -9 mRNAs in SW837 Colon Cancer Cells

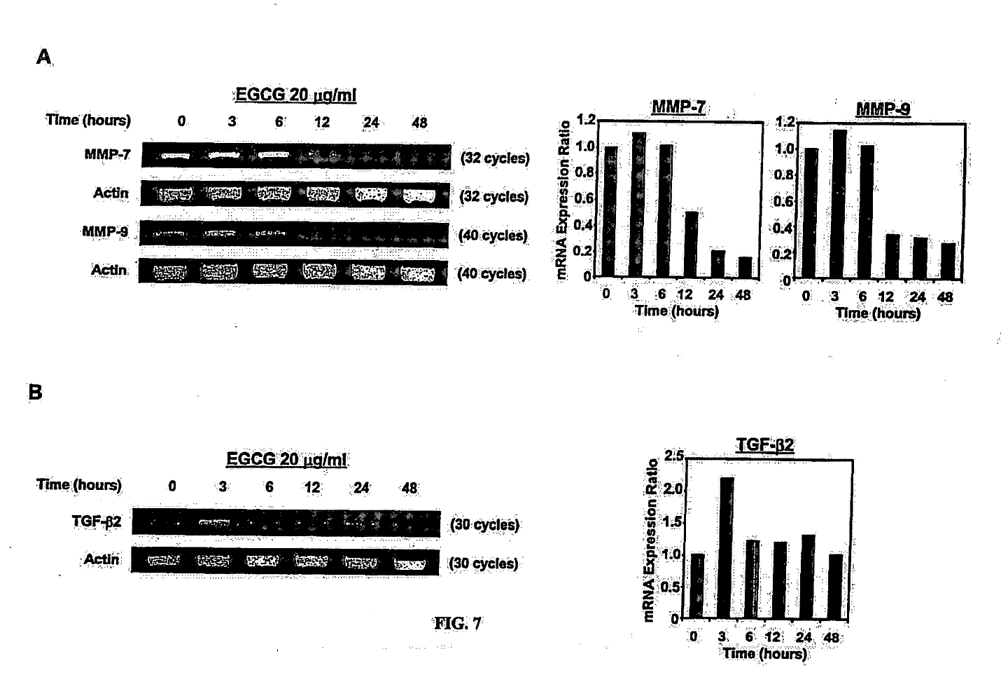

The time-dependent effects of EGCG on expression of MMP-7 and MMP-9 mRNA was measured by treating SW837 human colon cancer cells with 20 μg/mL EGCG. Results show that there was a marked decrease in expression of the cellular levels of both MMP-7and MMP-9 mRNA at 12 hours, and an additional decrease at 24 to 48 hours, as shown in FIG. 7A.

### 6.24 Example 24

### Effects of EGCG on Expression of TGF-β2 mRNA in SW837 Colon Cancer Cells

Is is indicated that inhibition of the growth of human breast cancer cells by TGF□2 is associated with induction of increased levels of IGFBP-3 mRNA and protein, as set forth in Example 23 and FIG. 7. Therefore, as a possible mechanism by which EGCG caused an increase in the levels of both IGFBP-3 protein and mRNA in SW837 colon cancer cells, the effects of EGCG on cellular levels of TGF-□2 mRNA were examined using semiquantitative RT-PCR analysis. Results indicate that the level of TGF-□2 mRNA was transiently increased at 3 hours after treatment with 20 μg/mL of EGCG, as shown in FIG. 7B.

### 6.25 Example 25

### Effects of EGCG on the Production of IGF-1, IGF-2 and IGFBP-3 in HepG2 Human Liver Cancer Cells

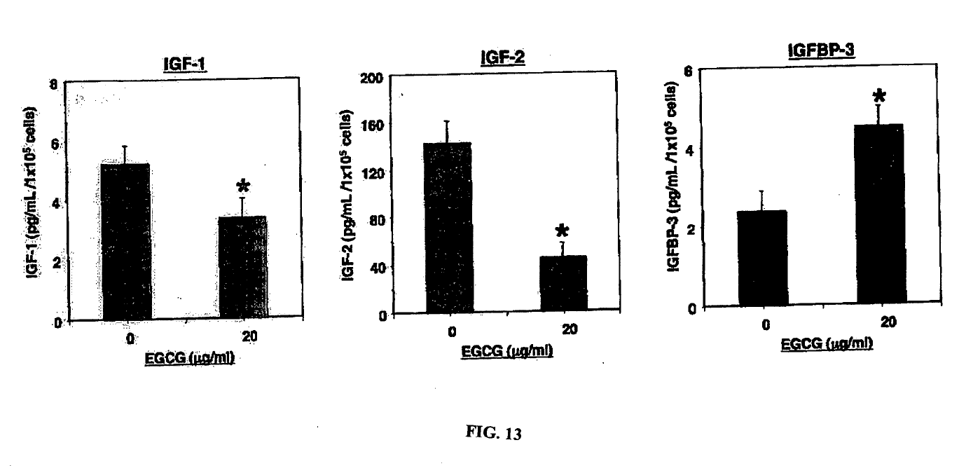

Using the MTT cell proliferation assay described in Example 21, and substituting HepG2 human liver cancer cells in place of SW837 cells, the effects of EGCG (20 μg/mL), an illustrative Synergistic Polyphenol Compound, on the production of IGF-1, IGF-2 and IGFBP-3 was determined. Results indicate that EGCG at 20 μg/mL inhibited the growth of HepG2 cells with an IC₅₀ of 21 μg/mL, similar to results obtained with colon cancer cells. The treatment of HepG2 cells with EGCG also inhibited production of the growth factors IGF-1 and IGF-2, but increased the production of IGFBP-3, as shown in FIG. 13. Accordingly, the Synergistic Polyphenol Compounds are useful for treating or preventing liver cancer.

### 6.25 Example 25

### Effect of EGCG and Polyphenone E on Ordered Membrane Domains in HT29 Colon Cancer Cells

Pretreatment with DilC₁₆

HT29 human colon cancer cells were treated with the fluorescent lipid analog DilC₁₆, which preferentially incorporates into ordered domains in the plasma membrane. Subsequent treatment of the cells with EGCG, an illustrative Synergistic Polyphenol Compound, or Polyphenone E, an illustrative Synergistic Polyphenol Compound, resulted in a marked increase in the sensitivity of the plasma membrane to extraction using cold Triton X-100, indicating that liquid ordered domains are decreasing.

Pretreatment with a Synergistic Polyphenol Compound or Composition

HT29 cells were pretreated with EGCG or Polyphenone E and both compounds were shown to inhibit subsequent incorporation of DilC₁₆ into the plasma membrane, this result being indicative of a change in membrane organization, induced by the EGCG or Polyphenone E.

These effects were detected with as little as 2 mg/mL of EGCG, and within 5 minutes. Phosphorylation of EGFR, ERK1, ERK2, and AKT were suppressed in cells treated with methyl-β-cyclodextrin, which depletes plasma membrane cholesterol. However, filipin staining indicated that the effects of EGCG on plasma membrane properties are not associated with cholesterol depletion. In addition, the inhibitory effect of EGCG on phosphorylation of EGFR was not blocked by the addition of cholesterol on the growth medium. These findings provide evidence that EGCG can alter membrane domain organization in colon cancer cells ant that this is not due to cholesterol depletion. It was noted that pre-treatment of the cells with EGCG or Poly E inhibited the binding of Alexa 488-labeled EGF to the cell surface receptor EGFR. Accordingly, these data suggest that the Synergistic Polyphenol Compounds and Compositions inhibit EGF binding, and subsequent activation of the EGFR and downstream signaling pathway by disrupting membrane domain organization.

The present invention is not to be limited in scope by the specific embodiments disclosed in the examples, which are intended as illustrations of a few aspects of the invention and any embodiments that are functionally equivalent are within the scope of this invention. Indeed, various modifications of the invention in addition to those shown and described herein will become apparent to one skilled in the art and are intended to fall within the scope of the appended claims.

What is claimed is:

1. A composition comprising two or more compounds or pharmaceutically acceptable salts thereof, wherein the compounds are selected from a catechin, including but not limited to (−)-epicatechin, (−)-epicatechin gallate, (−)-gallocatechin gallate, (−)-epigallocatechin and (−)-epigallocatechin gallate; a phenolic acid, including but not limited to gallic acid, caffeic acid and ellagic acid; a bioflavanoid, including but not limited to an anthocyanin, apigenin, and quercetin; and a complex polyphenol, including but not limited to, a tannin and a lignan, and any combination thereof, and wherein the amounts of the two or more compounds are together synergistically effective to treat or prevent colorectal cancer or liver cancer.

2. The composition of claim 1, wherein the compounds are selected from (+)-polyphenol, (−)-epicatechin, (−)-epicatechin gallate, (−)-gallocatechin gallate, (−)-epigallocatechin and (−)-epigallocatechin gallate.

3. The composition of claim 1 comprising (−)-epigallocatechin gallate.

4. The composition of claim 2 further comprising (−)-epigallocatechin.

5. The composition of claim 2 further comprising (−)-epicatechin gallate.

6. The composition of claim 2 further comprising (−)-gallocatechin gallate.

7. The composition of claim 2 further comprising (−)-epicatechin.

8. The composition of claim 2 further comprising (+)-polyphenol.

9. The composition of claim 1, wherein the two or more compounds are together synergistically effective to treat or prevent colorectal cancer.

10. The composition of claim 1, wherein the two or more compounds are together synergistically effective to treat or prevent liver cancer.

11. The composition of claim 2 consisting essentially of (−)-epigallocatechin gallate and (−)-epigallocatechin.

12. The composition of claim 2 consisting essentially of (−)-epigallocatechin gallate and (−)-epicatechin gallate.

13. The composition of claim 2 consisting essentially of (−)-epigallocatechin gallate and (−)-gallocatechin gallate.

14. The composition of claim 2 consisting essentially of (−)-epigallocatechin gallate and (−)-epicatechin.

15. The composition of claim 2 consisting essentially of (−)-epigallocatechin gallate and (+)-polyphenol.

16. The composition of claim 1 or 2 wherein one or more of the compounds are in isolated form.

17. The composition of claim 14, wherein the ratio of (−)-epicatechin to (−)-epigallocatechin gallate to is from about 5 to about 2.

18. The composition of claim 14, wherein the ratio of (−)-epicatechin to (−)-epigallocatechin gallate to is from about 5 to about 1.

19. A method for treating or preventing colorectal cancer or liver cancer in a subject, the method comprising administering to the subject an effective amount of the composition of claim 1.

20. The method of claim 19, wherein the composition of claim 1 comprises (−)-epigallocatechin gallate.

21. The method of claim 19, wherein the composition further comprises (−)-epicatechin

22. A method for treating colorectal cancer or liver cancer in a subject, the method comprising:
(a) administering to the subject a first compound or pharmaceutically acceptable salt thereof, selected from a catechin, including but not limited to (−)-epicatechin, (−)-epicatechin gallate, (−)-gallocatechin gallate, (−)-epigallocatechin and (−)-epigallocatechin gallate; a phenolic acid, including but not limited to gallic acid, caffeic acid and ellagic acid; a bioflavanoid, including but not limited to an anthocyanin, apigenin, and quercetin; and a complex polyphenol, including but not limited to, a tannin and a lignan, and any combination thereof; and
(b) administering to the subject a second compound or pharmaceutically acceptable salt thereof, selected from a catechin, including but not limited to (−)-epicatechin, (−)-epicatechin gallate, (−)-gallocatechin gallate, (−)-epigallocatechin and (−)-epigallocatechin gallate; a phenolic acid, including but not limited to gallic acid, caffeic acid and ellagic acid; a bioflavanoid, including but not limited to an anthocyanin, apigenin, and quercetin; and a complex polyphenol, including but not limited to, a tannin and a lignan, and any combination thereof,
wherein the first and second compounds are different and wherein the amounts of the first and second compounds administered are together synergistically effective to treat or prevent colorectal cancer or liver cancer.

23. The method of claim 22, wherein the first compound is (−)-epigallocatechin gallate.

24. The method of claim 23, wherein the second compound is (−)-epicatechin.

25. The method of claim 24, wherein the ratio of the amount of (−)-epicatechin administered to the amount of (−)-epigallocatechin gallate administered to is about 5 to about 2.

26. The method of claim 23, wherein the ratio of the amount of (−)-epicatechin administered to the amount of (−)-epigallocatechin gallate administered to is about 5 to about 1.

27. The method of claim 22 wherein the first compound is administered prior to the second compound.

28. The method of claim 22 wherein the first compound is administered at some time after the second compound.

29. The method of claim 22 wherein the first compound and second compound are administered concurrently.

30. The method of claim 22 wherein at least one of the first compound and the second compound are in isolated form.

31. The method of claim 22, further comprising the administration of another anticancer agent.

32. The method of claim 31, wherein the other anticancer agent is taxol or 5-fluorouracil.


SEQUENCE LISTING (12 sequences)

SEQ ID NO: 1 - DNA, 20 bp, Artificial
  Synthetic oligonucleotide
gttcccacccatgtcaaaac

SEQ ID NO: 2 - DNA, 20 bp, Artificial
  Synthetic oligonucleotide
gccctcgcttatgatctgtc

SEQ ID NO: 3 - DNA, 20 bp, Artificial
  Synthetic oligonucleotide
gctacagcatgcagagcaag

SEQ ID NO: 4 - DNA, 20 bp, Artificial
  Synthetic oligonucleotide
acgcccttgtttcagaaatg

SEQ ID NO: 5 - DNA, 20 bp, Artificial
  Synthetic oligonucleotide
gaaggtgaagatgcacacca

SEQ ID NO: 6 - DNA, 20 bp, Artificial
  Synthetic oligonucleotide
caggtaactcgtgcagagca

SEQ ID NO: 7 - DNA, 20 bp, Artificial
  Synthetic oligonucleotide
gagtgccagatgttgcagaa

SEQ ID NO: 8 - DNA, 20 bp, Artificial
  Synthetic oligonucleotide
tggggatctccatttccata

SEQ ID NO: 9 - DNA, 20 bp, Artificial
  Synthetic oligonucleotide
ggcgctcatgtaccctatgt

SEQ ID NO: 10 - DNA, 20 bp, Artificial
  Synthetic oligonucleotide
tcaaagaccgagtccagctt

SEQ ID NO: 11 - DNA, 20 bp, Artificial
  Synthetic oligonucleotide
tgctgttcatgaatggcttc

SEQ ID NO: 12 - DNA, 20 bp, Artificial
  Synthetic oligonucleotide
taagctcaggaccctgctgt